(19) United States
(12) Patent Application Publication
(10) Pub. No.: US 2022/0111361 A1
(43) Pub. Date: Apr. 14, 2022

(54) CO2 HYDROGENATION AND FISCHER-TROPSCH TO OLEFINS CATALYST

(71) Applicants: UNIVERSITY OF PITTSBURGH - OF THE COMMONWEALTH SYSTEM OF HIGHER EDUCATION, Pittsburgh, PA (US); NATIONAL ENERGY TECHNOLOGY LABORATORY (NETL), Pittsburgh, PA (US)

(72) Inventors: Sittichai NATESAKHAWAT, Pittsburgh, PA (US); Christopher MATRANGA, Pittsburgh, PA (US); Douglas KAUFFMAN, Pittsburgh, PA (US); Congjun WANG, Bethel Park, PA (US); Yunyun ZHOU, South Park, PA (US)

(73) Assignees: UNIVERSITY OF PITTSBURGH - OF THE COMMONWEALTH SYSTEM OF HIGHER EDUCATION, Pittsburgh, PA (US); NATIONAL ENERGY TECHNOLOGY LABORATORY (NETL), Pittsburgh, PA (US)

(21) Appl. No.: 17/420,779

(22) PCT Filed: Jan. 13, 2020
(86) PCT No.: PCT/US2020/013297

§ 371 Date: Jul. 6, 2021

Related U.S. Application Data
(60) Provisional application No. 62/792,051, filed on Jan. 14, 2019.

(51) Int. Cl. B01J 23/745; B01J 21/18; B01J 27/22; B01J 37/18; B01J 37/02; B01J 37/08; C07C 1/04

(52) U.S. Cl. CPC B01J 23/745; B01J 21/18; B01J 27/22; B01J 37/18; C07C 2527/22; B01J 37/0221; B01J 37/088; C07C 1/044; B01J 37/0207

(57) ABSTRACT

The invention relates to nanocatalysts composed of iron oxide nanoparticles supported on porous interconnected carbon nanosheets (CNS) fabricated from the carbonization of potassium citrate, that are remarkably active for CO₂ hydrogenation and Fischer-Tropsch to Olefins (FTO) synthesis, as well as a method for directly converting CO₂ and H₂ to C₂-C₄ olefins and direct FTO synthesis.

### CROSS-REFERENCE TO RELATED APPLICATION

This application claims priority under 35 U.S.C. § 119(e) to U.S. Provisional Patent Application Ser. No. 62/792,051, filed Jan. 14, 2019, entitled “CO₂ HYDROGENATION AND FISCHER-TROPSCH TO OLEFINS CATALYST”, which is herein incorporated by reference.

### GOVERNMENT SUPPORT

This invention was made with government support under DE-FE-00040001634917 awarded by the Department of Energy (DOE). The government has certain rights in the invention.

### FIELD OF THE INVENTION

The present invention relates to nanocatalysts composed of iron oxide nanoparticles supported on porous interconnected carbon nanosheets (CNS) fabricated from the carbonization of potassium citrate, that are remarkably active for C₀₂ hydrogenation and Fischer-Tropsch to Olefins (FTO) synthesis.

### BACKGROUND OF THE INVENTION

Light olefins (C₂-C₄) are important building blocks in the chemical industry. They serve as raw materials for the production of chemicals, plastics, solvents, cosmetics, drugs, detergents and the like. They are among the highest production volume petrochemical products worldwide. Traditionally, light olefins are produced from steam cracking and catalytic cracking of naphtha, gas oil or light alkanes. However, these processes are extremely energy intensive with significant CO₂ emissions. Thus, these processes are neither economical nor environmentally friendly approaches.

There has been significant interest in reducing the dependence on petroleum feedstocks for producing light olefins. Two promising alternative processes are (i) catalytic C₀₂ hydrogenation and (ii) Fischer-Tropsch to Olefins (FTO) synthesis. Both of these processes provide a direct route (absent an intermediate step) to produce light olefins directly from a mixture of CO₂ and H₂ or from syngas (CO and H₂), which can be derived from coal, natural gas, petroleum, or biomass. A direct route is more sustainable and potentially economically profitable. To this end, catalysts such as Fe- and Co-based Fischer-Tropsch to Olefins (FTO) catalysts and hybrid oxide-zeolite catalysts have been developed. Some of the catalysts have demonstrated remarkable olefin selectivity exceeding predictions from the Anderson-Schulz-Flory distribution, which describes the ideal relative ratios of different hydrocarbons in a polymerization process.

However, both of these alternative processes also have significant challenges associated with them, such as weak catalyst activity, poor stability and limited product selectivity. For example, for CO₂ hydrogenation, in most cases, C₁ molecules such as CO, CH₄, and CH₃OH are the main products and the selectivity to olefins tends to be low. For FTO processes, a variety of C₁ to C₅+ molecules are typically produced.

Additionally, iron-based catalysts have received increasing attention for FTO synthesis. Despite recent advances, challenges such as poor stability and/or high methane and CO₂ production still persist. One particular difficulty is the sintering and deactivation of catalyst nanoparticles during reaction. If the interaction between catalyst and support is strong, it will limit sintering. However, strong interaction can also inhibit catalyst activity. Consequently, there has been a focus on catalyst support materials that can promote the reducibility and carburization of catalysts and minimize aggregation and fragmentation. Carbon-based support materials have been reported as promising. For example, one of the best performing FTO catalysts in the art is Na- and S-promoted iron oxide on carbon nanofibers. Compared with metal oxide supports, weak interaction between iron oxide catalyst particles and the carbon nanofiber support facilitates catalyst activation while maintaining structural stability.

Thus, there is a need in the art to design and develop nanocatalysts that provide one or more of acceptable stability, catalyst activity and light olefin selectivity, as well as methods for directly converting CO₂ and H₂ to C₂-C₄ olefins and direct FTO synthesis.

### SUMMARY OF THE INVENTION

An object of the present invention is to develop a novel nanocatalyst that includes iron oxide nanoparticles and a support for the iron oxide nanoparticles, which includes a carbon nanosheet structure composed of a plurality of porous interconnected carbon nanosheets and a promoter K substantially uniformly embedded in the carbon nanosheets.

The iron oxide nanoparticles are unexpectedly active for CO₂ hydrogenation and Fischer-Tropsch to Olefins synthesis.

The nanocatalyst may be reusable repeatedly with very little degradation in catalytic performance over 500 hours of cumulative time on stream (TOS).

In one aspect, the invention provides a nanocatalyst that includes a support structure including a plurality of porous interconnected carbon nanosheets, and a potassium promoter embedded in the carbon nanosheets; and plurality of iron oxide nanoparticles supported on the support structure.

The iron oxide nanoparticles may include Fe₃O₄.

Another object of the present invention is to provide a novel method of directly converting CO₂ and H₂ to C₂-C₄ olefins and direct FTO synthesis. The method includes forming a nanocatalyst, which includes depositing iron oxide nanoparticles on a support, and preparing the support that includes forming a carbon nanosheet structure by interconnecting a plurality of porous carbon nanosheets, and uniformly embedding a promoter K in the carbon nanosheets; reducing the iron oxide nanoparticles to metallic iron; and transforming the metallic iron into iron carbide.

The forming of the carbon nanosheet structure can include carbonization of potassium citrate.

In another aspect, the invention provides a method of forming a nanocatalyst. The method includes preparing a support structure that includes interconnecting a plurality of porous carbon nanosheets; and embedding a potassium promoter in the carbon nanosheets; depositing a plurality of iron oxide nanoparticles on the support structure; reducing the plurality of iron oxide nanoparticles to metallic iron; and transforming the metallic iron into iron carbide.

The depositing step can include an iron precursor. The iron precursor may include ammonium iron citrate.

The preparing step may include carbonization of potassium citrate.

The reducing and transforming steps may include reducing Fe₃O₄ nanoparticles to metallic iron nanoparticles and subsequently, transforming to active Fe₅C₂. In certain embodiments, the reducing step includes exposing the Fe₃O₄ nanoparticles to H₂ activation, and the transforming step includes exposing the metallic iron nanoparticles to syngas.

The nanocatalyst can be used in CO₂ hydrogenation and Fischer-Tropsch to Olefins synthesis. Further, the nanocatalyst is reusable repeatedly without degradation in catalytic performance for at least 500 hours of cumulative TOS.

In yet another aspect, the invention includes a method of preparing C₂-C₄ olefins. The method includes fabricating a nanocatalyst including preparing a support structure, which includes obtaining a plurality of carbon nanosheets; interconnecting the plurality of carbon nanosheets; carbonizing a potassium precursor; and dispersing potassium promoter throughout the plurality of carbon nanosheets; and depositing a plurality of iron oxide nanoparticles on the support structure; initiating H₂ activation for reducing the plurality of iron oxide nanoparticles to metallic iron nanoparticles; and exposing the metallic iron nanoparticles to carburization for transforming the metallic iron into active iron carbide.

### BRIEF DESCRIPTION OF THE DRAWINGS

A full understanding of the invention can be gained from the following description of the preferred embodiments when read in conjunction with the accompanying drawings.

FIG. 1 includes views (a), (b), (c) and (d), wherein 1(a) is a scanning electron microscopy (SEM); 1(b) is transmission electron microscopy (TEM) images of the FeₓO_{y}/CNS catalyst, (inset) size distribution of FeₓO_{y} nanoparticles from analysis of >800 nanoparticles; 1(c) is a XRD pattern of FeₓO_{y}/CNS catalyst compared with the standard reference of Fe₃O₄ (PDF 01-076-1849); and 1(d) is a high resolution TEM (HRTEM) image of FeₓO_{y} nanoparticles showing lattice fringes consistent with the Fe₃O₄ phase;

FIG. 2 includes views (a) and (b), wherein 2(a) is a plot of the catalytic performance of FeₓO_{y}/CNS with iron time yield (FTY) (circles, right y axis), CO conversion (%, triangles, lefty axis); and 2(b) is a plot of light olefins selectivity (wt. %, squares, left y axis) and olefin to paraffin ratio, O/P, (diamonds, right y axis) as a function of time on stream (TOS) under reaction condition of 350° C., 20 bar, H₂/CO=1 and WHSV=30,000 cm³(STP)/(g_{cat}·h);

FIG. 3 includes views (a) and (b), wherein 3(a) is a TEM image of fresh FeₓO_{y}/carbon nanotube (CNT) catalyst, (inset) particle size distribution; and 3(b) is a plot of FTY (circles, left y-axis) and O/P ratio (diamonds, right y-axis) of FeₓO_{y}/CNT for FTO as a function of time on stream;

FIG. 4 includes views (a), (b), (c) and (d), wherein 4(a) is a TEM image of spent FeₓO_{y}/CNS catalysts, (inset) particle size distribution of spent iron nanoparticles based on >500 nanoparticles; 4(b) is a HRTEM image of an isolated iron carbide/iron oxide core/shell nanoparticle with lattice spacing in the core consistent with that of Fe₅C₂; 4(c) shows ⁵⁷Fe Mössbauer spectra (hashed lines) of the fresh FeₓO_{y}/CNS catalyst with the overall spectral simulation (solid line) and a magnetic subcomponent simulation representing the ferrous sites of Fe₃O₄ (solid line); and 4(d) shows the spent FeₓO_{y}/CNS catalyst with the overall spectral simulation (solid line) as well as the spectral simulation representing x-Fe₅C₂ (solid line) and an additional iron carbide phase FeₓC (solid line); the arrows indicate the spectral component similar to the iron oxide component in the fresh catalyst;

FIG. 5 includes views (a) and (b) including Fourier transformed Fe K-edge extended x-ray absorption fine structure (EXAFS) data of fresh and reacted 5(a) FeₓO_{y}/CNS and 5(b) FeₓO_{y}/CNT samples at TOS=0 h (fresh), 0.5 h, 1 h, 2 h, 3 h, 4 h, 7 h and 10 h in H₂/CO (1:1) syngas at 20 bar and 350° C.; the inset shows the corresponding EXAFS spectra in k-space; the solid lines represent experimental data, and the circles are fitted spectra; the vertical dashed lines indicate the feature of Fe—Fe coordination from Fe₅C₂;

FIG. 6 includes views (a) and (b), wherein 6(a) is the comparison of the evolution of the coordination number (CN) of Fe—Fe scattering from Fe₅C₂ composition in the FeₓO_{y}/CNS (circles) and FeₓO_{y}/CNT (squares) catalysts as a function of TOS; and 6(b) is Fourier transformed Fe K-edge EXAFS spectra of H₂ reduced FeₓO_{y}/CNS and H₂ reduced FeₓO_{y}/CNT catalysts; the most significant distinction between the two spectra is the two additional peaks at 2.2 Å and 4.4 Å observed in reduced FeₓO_{y}/CNS, which correspond to the Fe—Fe bonds from metallic iron; in contrast, the reduced FeₓO_{y}/CNT catalyst is mostly comprised of oxidized Fe species;

FIG. S1 is x-ray diffraction (XRD) pattern of carbon nanosheets;

FIG. S2 includes views (a) and (b) wherein S2(a) and S2(b) show additional high resolution transmission electron microscopy (HRTEM) images of fresh FeₓO_{y}/CNS catalysts;

FIG. S3 includes views (a) and (b) wherein S3(a) is Raman spectrum and S3(b) is x-ray photoelectron spectroscopy (XPS) C is spectrum and fitting analysis of the fresh FeₓO_{y}/CNS catalyst;

FIG. S4 includes views (a), (b), (c) and (d) wherein is S4(a) is scanning transmission electron microscopy (STEM) image and energy dispersive x-ray analysis (EDX) mapping of fresh FeₓO_{y}/CNS catalysts showing the distribution of Fe, O and K elements, i.e., S4(b), S4(c) and S4(d), respectively, on the CNS support (scale bars, 100 nm);

FIG. S5 is in situ XRD patterns of fresh FeₓO_{y}/CNS catalyst under 4% H₂/Ar (20 SCCM) reduction while heating from 25° C. to 400° C.; temperature ramp rate was 5° C./min; the temperatures were held for 10 min to reach the stable state; the 10 scans at 400° C. were collected with 30 min interval; the scans were taken with a step size of 0.017° and scan rate of 200 s/step; Al₂O₃ signal was from the sample holder;

FIG. S6 is XRD pattern of FeₓO_{y}/CNT catalyst and blank CNT with standard reference of Fe₃O₄ (PDF 01-076-1849);

FIG. S7 includes views (a) and (b) wherein S7(a) is Raman spectrum and S7(b) is C is XPS spectrum of FeₓO_{y}/CNT catalyst;

FIG. S8 includes views (a) and (b) wherein S8(a) is CO conversion and S8(b) is O/P ratio of FeₓO_{y}/CNS and FeₓO_{y}/CNT catalysts for FTO as a function of time on stream;

FIG. S9 includes views (a), (b), (c) and (d) wherein S9(a) is a high-angle annular dark field-STEM image of an iron-based nanoparticle in the spent FeₓO_{y}/CNS catalyst; EDX elemental mapping images of iron in S9(b), carbon in S9(c) (dashed line shows the outline of the Fe₅C₂ core) and oxygen elements in S9(d), because of the carbon support used for the catalysts, additional C signal is seen in areas outside of the nanoparticle;

FIG. S10 is laboratory based Fe 2p XPS spectra of spent FeₓO_{y}/CNS catalyst as a function of sputtering time; the Fe₅C₂ feature at 708.0 eV becomes increasingly pronounced as the surface oxide layer is removed by sputtering; the features at 711.3 eV and 710.4 eV correspond to Fe³⁺ and Fe²⁺ species, respectively; the very weak Fe features in the first spectrum at 0s are likely due to the surface coating of catalysts with contaminants such as carbon; as sputtering removed the surface coating, the spectral features become clearer;

FIG. S11 includes views (a), (b), (c) and (d) showing Fe K edge x-ray absorption near edge structure (XANES) profiles of FeₓO_{y}/CNS (in S11(a)) and FeₓO_{y}/CNT (in S11(b)) fresh and reacted samples at different TOS as indicated by the legend; iron oxides reference standards including Fe₃O₄, Fe₂O₃ and FeO are also shown in S11(a); S11(c) and S11(d) show the zoom-in and overlaid spectra in S11(a) and S11(b), respectively, illustrating the spectral changes in pre-edge and white line as a function of TOS; the arrow at pre-edge shows the appearance and evolution of features associated with iron carbide phase, and the arrow at line points to the intensity decrease of oxide phase with TOS;

FIG. S12 is Fourier transformed Fe K-edge EXAFS data of fresh FeₓO_{y}/CNS samples; the inset shows the corresponding EXAFS spectra of these samples in k-space; the lines represent experimental data, and circles are fitted spectra; a single model magnetite Fe₃O₄ is applied to fit the spectra of the fresh catalysts (Table S2);

FIG. S13 includes views (a) and (b) showing relative amplitudes of iron oxide and iron carbide phases indicated by EXAFS coordination numbers (CN) of neighboring atoms in reacted FeₓO_{y}/CNS (in S13(a)) and FeₓO_{y}/CNT (in S13(b)) samples with TOS from 0.5 h to 10 h; contributions from neighboring Fe-O coordination shell from Fe₃O₄, and Fe—C and Fe—Fe neighboring shells from Fe₅C₂ crystal structures are presented;

FIG. S14 includes views (a), (b), (c) and (d) showing high-angle annular dark field-STEM image of S14(a) a Fe-based nanoparticle in reduced FeₓO_{y}/CNS catalyst showing a core-shell structure; EDX mapping images of Fe, O and C in S14(b), S14(c), S14(d), respectively;

FIG. S15 is H₂-temperature programmed reduction (TPR) profiles of FeₓO_{y}/CNS and FeₓO_{y}/CNT catalysts; the inverse peak at 700° C. of FeₓO_{y}/CNS profile is due to the methanation of CNS support that typically occurred in the carbon supported iron catalysts; a lower onset reduction temperature˜200° C. and a larger reduction peak below 400° C. appear in the FeₓO_{y}/CNS catalyst; the reduction of FeₓO_{y} particles is less efficient on CNTs;

FIG. S16 is synchrotron XPS K 2p core-level spectra of fresh FeₓO_{y}/CNS catalysts;

FIG. X is a bar graph that shows CO₂ hydrogenation catalytic performance for a FeₓO_{y}/CNS catalyst, in accordance with certain embodiments of the invention;

FIG. Y is a bar graph that shows C₁-C₅ hydrocarbon product distribution from CO₂ hydrogenation, in accordance with certain embodiments of the invention; and

FIG. Z is a plot that shows CO₂ hydrogenation activity of the FeₓO_{y}/CNS catalyst as a function of time on stream (TOS), in accordance with certain embodiments of the invention.

### DETAILED DESCRIPTION OF THE INVENTION

The invention relates to nanocatalysts composed of iron oxide nanoparticles supported on carbon nanosheet. In certain embodiments, the carbon nanosheet includes a plurality of porous interconnected carbon nanosheets (CNS) fabricated from the carbonization of potassium citrate, that are remarkably active for CO₂ hydrogenation and Fischer-Tropsch to Olefins (FTO) synthesis. The inventors have found that FTO catalysts according to the invention have a very high iron time yield, e.g., 1882 μmol_{CO}/g_{Fe}·s, light olefins selectivity, e.g. 41%, and extended stability, e.g., over 100 hours of testing.

Detailed characterization of the inventive nanocatalysts has shown that the CNS support facilitates iron oxide, e.g., Fe₃O₄, reduction to metallic iron, leading to efficient transformation to an active iron carbide phase during FTO reaction. For example, the iron oxide catalyst is transformed to metallic iron nanoparticles during H₂ activation step and then to active iron carbide upon syngas exposure. The K-promoted CNS is effective to stabilize the metallic iron particles during H₂ reduction, which enhances formation of iron carbide under FTO reaction conditions, and the efficient carburization of the iron oxide/CNS catalyst results in high catalytic activity, selectivity and stability.

In certain embodiments, prior to FTO reaction, the Fe₃O₄/CNS catalyst is reduced in H₂ to form FeO and α-Fe phases, and further completely transformed to α-Fe metal. Under FTO conditions, the metal α-Fe is readily carburized and forms the active iron carbide species Fe₅C₂.

The invention includes the novel carbon nanosheet support material with embedded potassium (K) promoter. The K-promoted carbon nanosheets (CNS) are used as support for iron-based FTO catalysts which allows the formation of Fe₃O₄ nanoparticles on the surface. The K-promoted CNS catalyst effectively provides for the direct conversion of CO₂ and H₂ to light olefins, and also for direct FTO synthesis with extremely high activity.

The CNS supports are fabricated from the carbonization of potassium citrate as a K precursor that serves as an inexpensive carbon source with the added benefit of dispersing K promoter throughout the support. The catalyst demonstrates high activity and stability towards C₂-C₄ light olefins, and exhibits very high iron time yield (FTY) values, e.g., 1790-1990 μmol_{CO}/g_{Fe}'s for ˜100 hour time on stream (TOS). Furthermore, the catalyst can be repeatedly used while maintaining high activity for an extended period, e.g., at least 500 hours of cumulative TOS. Various characterization results have shown that the as-deposited iron oxide catalyst nanoparticles on K-promoted CNS are more readily reduced and stabilized as metallic iron after the initial H₂ activation compared with a control catalyst sample supported on carbon nanotubes (CNTs). In certain embodiments, ammonium iron citrate is used as a Fe precursor for depositing iron oxide nanoparticle catalysts on the CNS support. The more robust formation of metallic iron on CNS results in more efficient conversion in the subsequent FTO reaction to form highly active iron carbide. Also, the K embedded in CNS can enhance catalyst activity and selectivity. Moreover, K is a common promoter for a broad range of reactions and therefore, the inventive process for fabricating K-promoted CNS catalyst supports has broad utility.

In certain embodiments, the catalyst according to the invention can be effective to catalyze direct CO₂ hydrogenation to produce light olefins with up to 37% CO₂ conversion and 65% light olefins in the hydrocarbon distribution produced, as well as providing an extremely high iron time yield (FTY) of 1882 μmol_{CO}/g_{Fe}·s with 41% selectivity for light olefins and excellent stability (approximately 100 hours on stream) for FTO processes. The FTY value is 50 to 1300 times higher as compared to catalysts exhibiting similar light olefin selectivity known in the art. The catalyst according to the invention is highly active for 100 hours continuous time on stream and demonstrates very low degradation after repeated catalytic reaction cycles totaling 550 hours.

Since the novel carbon nanosheet (CNS) support material advantageously allows the formation of Fe₃O₄ phase of the iron oxide nanoparticles on the surface of the catalyst support, the transformation to highly active iron carbide Fe₅C₂ upon exposure to CO for FTO synthesis is facilitated. In contrast, similar synthesis parameters result in the Fe₂O₃ phase of iron oxide on other carbon support materials in the art. The advantages of Fe₃O₄ (as compared to Fe₂O₃) include (i) it is a highly active reverse water gas shift (RWGS) catalyst (CO₂+H₂=CO+H₂O), and (ii) it is more readily reduced to metallic iron compared to Fe₂O₃ which is essential to the subsequent conversion to form highly active Fe₅C₂ in the presence of CO for FTO synthesis (2nH₂+nCO=CₙH₂ₙ+nH₂O). Moreover, the novel CNS support offers advantages of 2D carbon-based catalyst support materials, and also contains a promoter, K, embedded uniformly in the CNS structure. The CNS support material has multiple functional groups such as carboxyl, carbonyl and hydroxyl groups. Without being bound by any particular theory, it is believed that these functional groups contribute to catalyst performance by anchoring and stabilizing supported iron oxide nanoparticles. Further, the ubiquitous and even distribution of K throughout the support contributes to the superior activity and performance of the nanocatalysts. It has been shown that using other carbon-based catalyst support, such as carbon nanotubes, or intentionally adding promoter K to other carbon support materials, will not achieve the high activity that is demonstrated by the CNS support material according to the invention.

Pursuant to the invention, the Fe₃O₄/CNS catalyst selectively converts CO₂ to C₂-C₄ olefins. Further, selectivity to the C₂-C₄ olefins is tuned by optimizing reaction parameters such as feed gas composition and space velocity. According to the invention, unwanted CH₄ production is suppressed by increasing H₂/CO₂ ratio of the feed gas or by lowering the reaction temperature.

It will be appreciated by those skilled in the art that changes could be made to the embodiments described above without departing from the broad inventive concept thereof. It is understood, therefore, that this invention is not limited to the particular embodiments disclosed and the following examples conducted, but it is intended to cover modifications that are within the spirit and scope of the invention.

### EXAMPLES

### Experimental Details

The following experimental details apply to the below Examples that were conducted.

Brunauer-Emmett-Teller (BET) surface area measurements of fresh catalysts were conducted in a Quantachrome Autosorb 1-C analyzer. Prior to N₂ isotherms at −196° C., the catalysts (approximately 40 mg) were degassed at 110° C. under vacuum overnight.

Raman spectra were obtained using a Horiba (LabRam HR-Evolution) spectrometer with a 633 nm laser excitation source. The laser excitation power was 67 mW and the filter size was 10%.

X-ray photoelectron spectroscopy (XPS) experiments were conducted with a PHI 5600ci spectrometer equipped with a hemispherical electron analyzer and A1 Kα (1486.6 eV) radiation source. The powder samples were mounted on double-sided carbon tapes for analysis. The core-shell structure of spent catalyst samples was analyzed with sputtering experiments conducted by Ar ion bombardment. The XPS data were collected with sputtering times of 30, 60, 90, 120, 180, and 300 seconds. Both C 1s and Fe 2p spectra were recorded. All binding energies were calibrated to the C is peak located at 284.6 eV.

Synchrotron-based XPS spectra of fresh, H₂ reduced, and spent FTO catalysts were collected at the beamline 23-ID-2 (IOS) at NSLS-II. The powder samples were pressed on an indium foil and the spectra were collected in UHV at room temperature using a SPECS Phoibos 150 NAP analyzer.

CO conversion, product selectivity, and FTY were determined/calculated as follows:

$$CO conversion ( \% ) = \frac{moles of CO , in - moles of CO , out}{moles of CO , in} \times 100$$

$$Selectivity of C product i ( wt \% , excluding CO 2 ) = \frac{[ ( molar flow rate ) ( MW ) ] i}{\sum [ ( molar flow rate ) ( MW ) ] i} \times 100$$

$$FTY ( µmolCO \text{/} g Fe \cdot s ) = \frac{molar flow rate of CO converted to HC products}{( catalyst weight ) ( \% Fe loading / 100 )}$$

The D and G bands (at ca. 1322 and 1580 cm-1) were clearly observed, corresponding to the structural disorder of the defects of carbon materials, and the vibration of the sp²-hybridized carbon atoms, respectively. The D/G band ratio is 1.13, indicating surface defect or structural disorder on the CNS support. Surface disorder and functional groups on the carbon catalyst support have been reported to be important factors affecting the FTO catalyst performance.

The C is spectra were deconvoluted into four peaks for FeₓO_{y}/CNS catalyst. Nonoxygenated C ring (284.4 eV), C in C—O (285.8 eV), carbonyl C (C═O, 287.2 eV) and carboxyl C (O═C—O, 289.4 eV) were applied to fit the spectra, with occupations of 68.1%, 20.7%, 6.1% and 5.1%, respectively. The C—O peak could be either C—OH or C—O—C functional groups, and cannot be distinguished from each other. The FeₓO_{y}/CNS sample clearly contained significant amounts of oxygen functional groups, which is beneficial to the homogeneous distribution of the supported iron nanoparticles.

The D and G bands at 1323 and 1590 cm⁻¹ exhibited a D/G band ratio of 1.38, indicating a slightly higher defective surface or structural disorder compared with the CNS support, which was likely responsible for the stronger interaction between CNT and FeₓO_{y} nanoparticles.

The C 1s spectra were deconvoluted into five peaks for FeₓO_{y}/CNT catalyst. Nonoxygenated C ring (284.4 eV), C—O (285.6 eV), carbonyl C (C═O, 286.9 eV), carboxyl C (O═C—O, 289.4 eV) and π-π* satellite at 291.2 eV were applied to fit the spectra, with occupations of 64.3%, 16.8%, 8.8%, 4.5% and 5.6% respectively. The FeₓO_{y}/CNT sample exhibited similar amounts of oxygen functional groups, which was also beneficial to the homogeneous distribution of the supported iron nanoparticles.

TABLE S1
Mössbauer simulation parameters of the fresh and spent FeₓO_{y}/CNS catalysts.

| | | δ | ΔEq | B_{hf} | Γ | | |
|---|---|---|---|---|---|---|---|
| Sample | Phase | (mm/s) | (mm/s) | (T) | (mm/s) | % | Comments |
| Fresh | FeₓO_{y}-I | 0.46 | 0 | 52.1 | 0.5 | 35 | Ferric sites, |
| FeₓO_{y}/ | | | | | | | Fe₂O₃ or Fe₃O₄ |
| CNS | FeₓO_{y}-II | 0.43 | 0 | 49.8 | 0.5 | 37 | Ferric sites, |
| | | | | | | | Fe₂O₃ or Fe₃O₄ |
| | FeₓO_{y}-III | 0.83 | −1.14 | 46.3 | 0.5 | 16 | Ferric sites, |
| | | | | | | | Fe₃O₄ |
| Spent | FeₓO_{y}-I | 0.46 | 0 | 52.1 | 0.5 | 6 | Ferric sites, |
| FeₓO_{y}/ | | | | | | | Fe₂O₃ or Fe₃O₄ |
| CNS | FeₓO_{y}-II | 0.43 | 0 | 49.8 | 0.5 | 6 | Ferric sites, |
| | | | | | | | Fe₂O₃ or Fe₃O₄ |
| | FeₓO_{y}-III | 0.45 | 0 | 46.5 | 0.5 | 5 | Ferric sites, |
| | | | | | | | Fe₂O₃ or Fe₃O₄ |
| | χ-Fe₅C₂-I | 0.39 | 0.09 | 25.5 | 0.4 | 30 | |
| | χ-Fe₅C₂-II | 0.33 | 0 | 21.8 | 0.55 | 27 | |
| | χ-Fe₅C₂-III | 0.33 | 0.05 | 10.6 | 0.5 | 15 | |
| | FeₓC | 0.36 | 0 | 18.3 | 0.3 | 10 | |

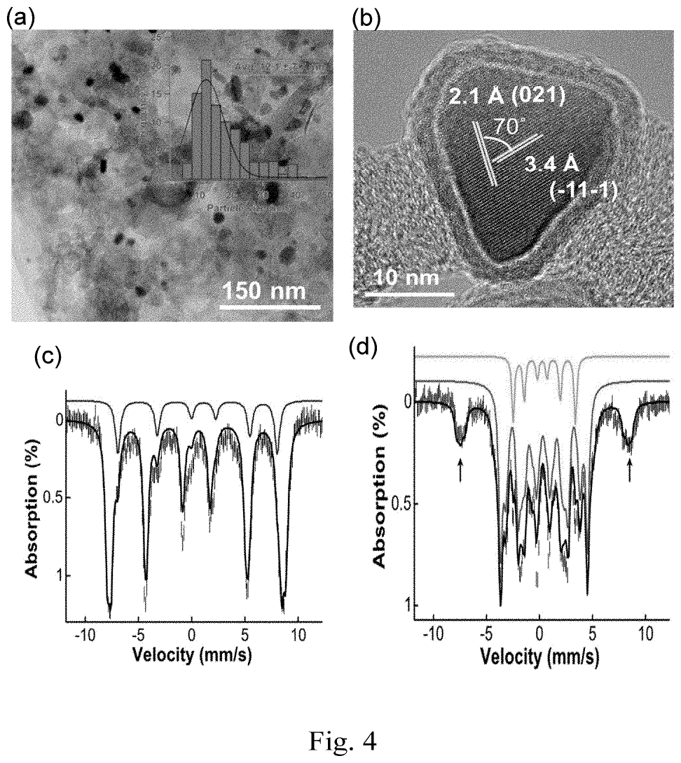

The Mössbauer spectrum of fresh FeₓO_{y}/CNS catalyst (FIG. 4c) can be simulated with three components (Table S1), two of them have ⁵⁷Fe hyperfine field value of ˜50 T and isomer shift value of ˜0.45 mm/s, which represent ferric sites in either Fe₂O₃ or Fe₃O₄. The third component has a ⁵⁷Fe hyperfine field value of 46.3 T, an isomer shift value of 0.83 mm/s, and represents typical ferrous sites in Fe₃O₄. The spent catalyst (FIG. 4d) has two components from ferric sites in either Fe₂O₃ or Fe₃O₄. These features accounted for 12% of the total Fe signal in the spent sample (the spectral features indicated by the arrows in FIG. 4d) and are also seen in the fresh, unactivated, catalysts. A third iron oxide feature was observed with a hyperfine field value of 46.5 T, an isomer shift of 0.45 mm/s, which accounted for 5% of the Fe signal; this feature was not seen in the fresh catalyst and likely arises from exposing the active catalyst to ambient after the reaction. The Mössbauer results also indicated that 72% of the Fe signal in the spent catalyst is composed of 3 Ψ—Fe₅C₂ features with hyperfine field values ranging from 10 T to 25 T and isomer shift values of ˜0.35 mm/s. There is an additional feature associated with an FeₓC phase (possibly Fe₂C or Fe_{2.2}C) that accounted for 10% of the Fe signal with a hyperfine field value of 18 T and an isomer shift of 0.36 mm/s.

The Fe K-edge XANES data indicated that the fresh FeₓO_{y}/CNS and FeₓO_{y}/CNT catalysts closely resemble the Fe₃O₄ phase rather than Fe₂O₃ and FeO phases (FIG. S11a,b), which is consistent with the TEM, XRD and Mössbauer results. The XANES spectra of samples after FTO reaction showed an absorption feature at 7111.2 eV similar to the iron carbide phase reported in the art and this feature continued to grow as a function of TOS (FIG. S11a,c). In addition to the distinct iron carbide feature, Fe₃O₄ phase was still present in the spent catalysts, reflected by the line at 7131.2 eV (FIG. S11c). The line intensity reduces and the spectral feature broadens as the reaction further proceeds. The line correlates with 1s to 4p transition and its intensity is an indicator of the degree of reduction and carburization of catalysts. The smaller line intensity is associated with higher degree of carburization and higher iron carbide content. A shift of edge rising position from 7126.4 eV of fresh catalyst to 7123.0 eV of spent catalyst after 10 h TOS implies a reduction process of iron species during FTO reaction. Consequently, the spent FeₓO_{y}/CNS catalysts appeared to be a mixture of iron carbide and oxide phases, which is consistent with the HRTEM, XPS and Mössbauer results. FeₓO_{y}/CNT catalysts showed similar conversion from iron oxide to iron carbide phase during FTO reaction (FIG. S11b,d). However, unlike the FeₓO_{y}/CNS catalysts, the changes in the XANES spectra were much less pronounced for the reacted FeₓO_{y}/CNT catalyst (FIG. S11b,d).

TABLE S2
Results of EXAFS data fitting analysis of fresh FeₓO_{y}/CNS sample. Fe₃O₄ (magnetite)
model was used for fresh FeₓO_{y}/CNS fitting analysis. Note that S₀² is fixed at 0.77.

| Samples | Model | Atoms | R(Å) | CN | σ²(Å²) | ΔE (eV) |
|---|---|---|---|---|---|---|
| Fresh | Tetrahedral Fe | Fe—O | 1.96 ± 0.00 | 2.8 ± 0.2 | 0.002 ± 0.001 | 3.38 ± 0.29 |
| FeₓO_{y}/CNS | | Fe—Fe | 3.38 ± 0.04 | 7.7 ± 1.4 | 0.022 ± 0.006 | |
| | | Fe—O | 3.54 ± 0.02 | 9.6 ± 1.9 | 0.022 ± 0.005 | |
| | | Fe—Fe | 3.70 ± 0.01 | 4.6 ± 1.2 | 0.009 ± 0.002 | |
| | Octahedral Fe | Fe—O | 2.11 ± 0.01 | 3.8 ± 0.4 | 0.018 ± 0.002 | 3.38 ± 0.29 |
| | | Fe—Fe | 3.00 ± 0.00 | 2.3 ± 0.6 | 0.005 ± 0.002 | |
| | | Fe—Fe | 3.50 ± 0.00 | 6.2 ± 0.8 | 0.004 ± 0.001 | |
| | | Fe—O | 3.76 ± 0.02 | 4.9 ± 2.4 | 0.016 ± 0.012 | |
| | Tetrahedral Fe | Fe—O | 1.86 | 4 | | |
| Fe₃O₄ | | Fe—Fe | 3.45 | 12 | | |
| (theoretical) | | Fe—O | 3.46 | 12 | | |
| | | Fe—Fe | 3.6 | 4 | | |
| | Octahedral Fe | Fe—O | 2.05 | 6 | | |
| | | Fe—Fe | 2.94 | 6 | | |
| | | Fe—Fe | 3.45 | 6 | | |
| | | Fe—O | 3.62 | 6 | | |

TABLE S3
Results of EXAFS data fitting analysis of fresh FeₓO_{y}/CNT sample. Fe₃O₄ (magnetite)
model is used for fresh FeₓO_{y}/CNT fitting analysis. Note that S₀² is fixed at 0.77.

| Samples | Model | Atoms | R(Å) | CN | σ²(Å²) | ΔE(eV) |
|---|---|---|---|---|---|---|
| Fresh | Tetrahedral Fe | Fe—O | 1.89 ± 0.00 | 2.02 ± 0.10 | 0.003 ± 0.001 | 1.24 ± 0.23 |
| FeₓO_{y}/CNT | | Fe—Fe | 3.47 ± 0.01 | 2.45 ± 0.70 | 0.005 ± 0.003 | |
| | | Fe—O | 3.45 ± 0.04 | 2.15 ± 0.61 | 0.012 ± 0.007 | |
| | | Fe—Fe | 3.97 ± 0.03 | 1.19 ± 0.62 | 0.010 ± 0.007 | |
| | Octahedral Fe | Fe—O | 2.04 ± 0.01 | 1.99 ± 0.11 | 0.003 ± 0.001 | 1.24 ± 0.23 |
| | | Fe—Fe | 2.97 ± 0.02 | 3.35 ± 0.30 | 0.011 ± 0.002 | |
| | | Fe—Fe | 3.14 ± 0.06 | 1.05 ± 0.65 | 0.021 ± 0.007 | |
| | | Fe—O | 3.46 ± 0.13 | 1.52 ± 0.46 | 0.013 ± 0.009 | |

In order to determine the relative quantities of iron oxides and iron carbides in these reacted catalysts, both Fe₃O₄ and Hägg carbide χ-Fe₅C₂ were employed as models for the EXAFS fitting analysis. χ-Fe₅C₂ was used instead of other carbides such as s-Fe_{2.2}C and O-Fe₃C because (a) HRTEM and Mössbauer spectroscopy analysis suggest Fe₅C₂ as the major carbide species in the spent catalysts, and (b) Hägg carbide x-Fe₅C₂ is a typical product formed between 250 and 350° C. under FT conditions, and the FTO reaction was carried out at 350° C. The Fe₅C₂ EXAFS spectrum showed two major peaks below 3 Å, corresponding to Fe—C (1.9 Å) and Fe—Fe (2.2 Å) scattering. Although the Fe—C peak overlaps with the Fe—O peak in iron oxides, it can be distinguished by EXAFS fitting analysis. FIG. S13a and Table S4 display the fitting results of the reacted FeₓO_{y}/CNS catalysts. The build-up of iron carbide phase as a function of TOS is illustrated by the coordination number (CN). The first Fe—C coordination shell grows gradually from 1.3 to 1.8 as TOS increases from 0.5 h to 10 h, due to the increasing contribution of Fe—C scattering from x-Fe₅C₂, whereas the coordination number of Fe—O from Fe₃O₄ slowly decreases from 3.2 to 2.0. There is a remarkable increase from 4.7 to 8.2 associated with the Fe—Fe coordination from x-Fe₅C₂ from TOS=0.5 h to 10 h. This is clear evidence of the carburization process of iron catalysts during FTO reaction. Debye-Waller factors (σ²) evaluated the crystal disorder and deviations from standard references. The rise of Debye-Waller factor of Fe—Fe shell from Fe₃O₄ from 0.5 h to 10 h originated from the gradual structural transformation from Fe₃O₄ to Fe₅C₂ (see details in Table S4).

The models of Fe₃O₄ and Fe₅C₂ and fitting procedure similar to those used for analyzing FeₓO_{y}/CNS spectra were applied for FeₓO_{y}/CNT. The detailed fitting results are in Table S3 and S5. Coordination number is a good indicator for phase composition change as a function of TOS (FIG. S13b). From 0.5 h to 10 h, the CN of Fe—O bond remained almost constant around 3.0, suggesting an insignificant change of Fe₃O₄ phase in the CNT supported nanoparticles. The lack of pronounced variation in reacted catalysts was also observed in the Fe—C and Fe—Fe coordination from the Fe₅C₂ phase (FIG. S13b). These results are in marked contrast to the significantly more pronounced conversion of Fe₃O₄ to Fe₅C₂ phase seen in CNS supported catalysts (FIG. S13a).

TABLE S4
Results of EXAFS data fitting analysis of reacted FeₓO_{y}/CNS
catalysts at different TOS. Hägg carbide and Fe₃O₄
(magnetite) are applied as the fitting models. Fitting intervals for k
and R space are 2.5~10 Å⁻¹, 1~3 Å, respectively. Global
parameters of S₀² 0.77 and energy shift ΔE 2.78 eV
are employed for the Fe—C and Fe—Fe (Fe₅C₂) paths from Hägg
carbide and Fe—O and Fe—Fe (Fe₃O₄) paths from
magnetite in all samples analysis.

| TOS | Atoms | R(Å) | CN | σ²(Å²) |
|---|---|---|---|---|
| 0.5 h | Fe—O | 1.98 ± 0.01 | 3.2 ± 0.6 | 0.010 ± 0.001 |
| | Fe—C | 1.98 ± 0.02 | 1.3 ± 0.3 | 0.003 ± 0.002 |
| | Fe—Fe (Fe₅C₂) | 2.69 ± 0.01 | 4.7 ± 0.5 | 0.014 ± 0.001 |
| | Fe—Fe (Fe₃O₄) | 2.99 ± 0.01 | 7.5 ± 1.0 | 0.022 ± 0.002 |
| 1 h | Fe—O | 1.98 ± 0.01 | 3.4 ± 0.3 | 0.011 ± 0.001 |
| | Fe—C | 1.97 ± 0.02 | 1.3 ± 0.3 | 0.003 ± 0.002 |
| | Fe—Fe (Fe₅C₂) | 2.69 ± 0.01 | 4.7 ± 0.5 | 0.014 ± 0.001 |
| | Fe—Fe (Fe₃C₄) | 2.98 ± 0.01 | 6.7 ± 0.9 | 0.022 ± 0.002 |
| 2 h | Fe—O | 1.98 ± 0.01 | 3.0 ± 0.2 | 0.010 ± 0.001 |
| | Fe—C | 1.97 ± 0.02 | 1.5 ± 0.2 | 0.003 ± 0.002 |
| | Fe—Fe (Fe₅C₂) | 2.69 ± 0.01 | 5.2 ± 0.7 | 0.015 ± 0.001 |
| | Fe—Fe (Fe₃C₄) | 2.98 ± 0.01 | 6.5 ± 1.1 | 0.024 ± 0.001 |
| 3 h | Fe—O | 1.98 ± 0.02 | 2.8 ± 0.3 | 0.010 ± 0.002 |
| | Fe—C | 1.97 ± 0.01 | 1.5 ± 0.3 | 0.003 ± 0.002 |
| | Fe—Fe (Fe₅C₂) | 2.69 ± 0.01 | 6.0 ± 0.9 | 0.016 ± 0.001 |
| | Fe—Fe (Fe₃O₄) | 2.98 ± 0.02 | 6.1 ± 1.2 | 0.024 ± 0.001 |
| 4 h | Fe—O | 1.98 ± 0.01 | 2.4 ± 0.2 | 0.010 ± 0.001 |
| | Fe—C | 1.97 ± 0.01 | 1.6 ± 0.2 | 0.003 ± 0.002 |
| | Fe—Fe (Fe₅C₂) | 2.69 ± 0.01 | 6.5 ± 0.7 | 0.016 ± 0.001 |
| | Fe—Fe (Fe₃O₄) | 2.97 ± 0.02 | 6.0 ± 1.1 | 0.028 ± 0.003 |
| 7 h | Fe—O | 1.98 ± 0.02 | 2.0 ± 0.3 | 0.007 ± 0.005 |
| | Fe—C | 1.95 ± 0.05 | 1.8 ± 0.2 | 0.002 ± 0.001 |
| | Fe—Fe (Fe₅C₂) | 2.67 ± 0.02 | 7.9 ± 0.9 | 0.016 ± 0.003 |
| | Fe—Fe (Fe₃O₄) | 2.96 ± 0.04 | 5.9 ± 1.6 | 0.029 ± 0.005 |
| 10 h | Fe—O | 2.00 ± 0.02 | 2.0 ± 0.3 | 0.008 ± 0.003 |
| | Fe—C | 1.95 ± 0.02 | 1.8 ± 0.2 | 0.002 ± 0.001 |
| | Fe—Fe (Fe₅C₂) | 2.69 ± 0.02 | 8.2 ± 0.9 | 0.018 ± 0.001 |
| | Fe—Fe (Fe₃O₄) | 2.94 ± 0.02 | 5.5 ± 1.2 | 0.030 ± 0.004 |

TABLE S5
Results of EXAFS data fitting analysis of reacted FeₓO_{y}/CNT
samples at different TOS. Hägg carbide and Fe₃O₄
(magnetite) are applied as the fitting models. Fitting intervals for k
and R space are 2.5~10 Å⁻¹, 1~3 Å, respectively. Global
parameters of S₀² 0.77 and energy shift ΔE 3.26 eV
are employed for the Fe—C and Fe—Fe (Fe₅C₂) paths from Hägg
carbide and Fe—O and Fe—Fe (Fe₃O₄) paths from
magnetite in all samples analysis.

| TOS | Atoms | R(Å) | CN | σ²(Å²) |
|---|---|---|---|---|
| 0.5 h | Fe—O | 1.99 ± 0.01 | 3.1 ± 0.2 | 0.009 ± 0.001 |
| | Fe—C | 1.93 ± 0.02 | 1.0 ± 0.1 | 0.002 ± 0.001 |
| | Fe—Fe (Fe₅C₂) | 2.61 ± 0.01 | 2.9 ± 0.3 | 0.013 ± 0.001 |
| | Fe—Fe (Fe₃O₄) | 2.95 ± 0.02 | 5.5 ± 1.4 | 0.030 ± 0.004 |
| 1 h | Fe—O | 1.99 ± 0.01 | 3.1 ± 0.2 | 0.009 ± 0.001 |
| | Fe—C | 1.94 ± 0.01 | 1.0 ± 0.2 | 0.002 ± 0.001 |
| | Fe—Fe (Fe₅C₂) | 2.61 ± 0.01 | 3.0 ± 0.4 | 0.013 ± 0.001 |
| | Fe—Fe (Fe₃O₄) | 2.94 ± 0.03 | 5.4 ± 1.1 | 0.032 ± 0.004 |
| 2 h | Fe—O | 1.99 ± 0.01 | 3.2 ± 0.3 | 0.009 ± 0.005 |
| | Fe—C | 1.93 ± 0.03 | 1.1 ± 0.2 | 0.002 ± 0.001 |
| | Fe—Fe (Fe₅C₂) | 2.61 ± 0.01 | 3.1 ± 0.4 | 0.013 ± 0.001 |
| | Fe—Fe (Fe₃O₄) | 2.94 ± 0.02 | 5.3 ± 1.3 | 0.031 ± 0.006 |
| 3 h | Fe—O | 1.99 ± 0.00 | 3.2 ± 0.2 | 0.009 ± 0.005 |
| | Fe—C | 1.93 ± 0.01 | 1.1 ± 0.2 | 0.002 ± 0.001 |
| | Fe—Fe (Fe₅C₂) | 2.61 ± 0.01 | 3.2 ± 0.3 | 0.013 ± 0.001 |
| | Fe—Fe (Fe₃O₄) | 2.93 ± 0.03 | 5.0 ± 1.2 | 0.032 ± 0.004 |
| 4 h | Fe—O | 1.96 ± 0.01 | 3.1 ± 0.4 | 0.011 ± 0.001 |
| | Fe—C | 2.01 ± 0.02 | 1.1 ± 0.2 | 0.008 ± 0.004 |
| | Fe—Fe (Fe₅C₂) | 2.61 ± 0.01 | 3.2 ± 0.3 | 0.013 ± 0.001 |
| | Fe—Fe (Fe₃O₄) | 2.93 ± 0.02 | 5.2 ± 1.1 | 0.032 ± 0.004 |
| 7 h | Fe—O | 1.96 ± 0.04 | 3.4 ± 0.2 | 0.011 ± 0.001 |
| | Fe—C | 1.97 ± 0.02 | 1.1 ± 0.2 | 0.006 ± 0.003 |
| | Fe—Fe (Fe₅C₂) | 2.61 ± 0.01 | 3.2 ± 0.3 | 0.015 ± 0.001 |
| | Fe—Fe (Fe₃O₄) | 2.94 ± 0.02 | 5.6 ± 1.2 | 0.032 ± 0.004 |
| 10 h | Fe—O | 1.96 ± 0.01 | 3.1 ± 0.3 | 0.011 ± 0.001 |
| | Fe—C | 1.96 ± 0.03 | 1.0 ± 0.3 | 0.006 ± 0.003 |
| | Fe—Fe (Fe₅C₂) | 2.63 ± 0.01 | 3.8 ± 0.4 | 0.015 ± 0.001 |
| | Fe—Fe (Fe₃O₄) | 2.93 ± 0.02 | 5.2 ± 1.3 | 0.032 ± 0.005 |

The electronic structure of fresh FeₓO_{y}/CNS catalysts was evaluated by means of synchrotron XPS technique. The K 2p spectra indicate the presence of potassium promoter in range of 292.5-298.5 eV. The significant asymmetry of K 2p region in fresh FeₓO_{y}/CNS material confirmed potassium species of partially oxidized potassium states. No plasmon features at higher binding energies for metallic K were found in the spectra.

Catalytic Performance for FTO Synthesis with FeₓO_{y}/CNS Catalysts

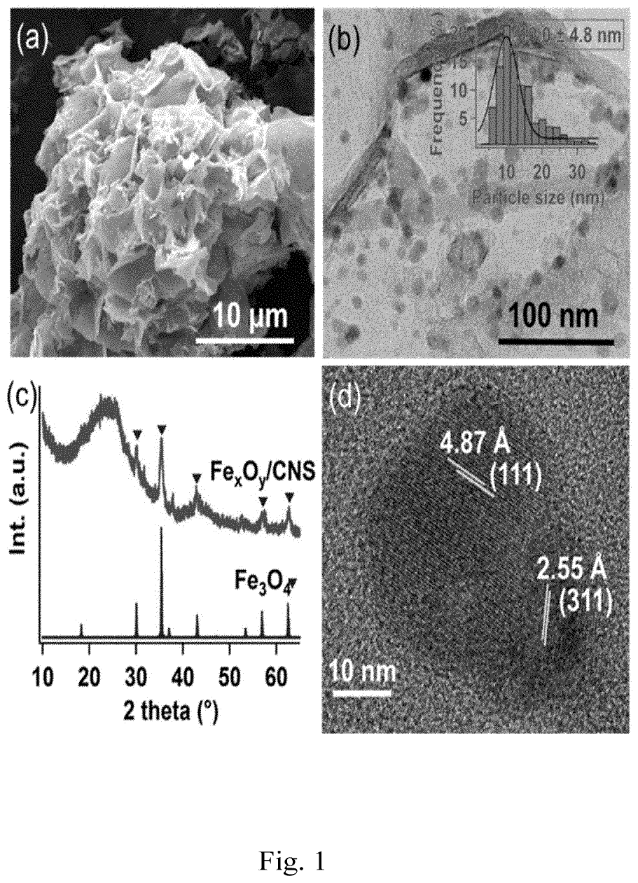

The as-synthesized iron oxide on interconnected carbon nanosheets (FeₓO_{y}/CNS) catalyst consisted of 10.0 nm±4.8 nm FeₓO_{y} nanoparticles well dispersed on CNS with a rose-like structure (FIG. 1a,b). XRD indicated the main crystal phase of FeₓO_{y} particles was Fe₃O₄ (FIG. 1c). The calculated size of Fe₃O₄ nanoparticles was 11 nm from the Scherer formula using the peak (311) at 35.4°, consistent with the TEM particle size analysis. The broad peak at 230 arises from the CNS (FIG. S1). The HRTEM images of the iron oxide nanoparticles in FIG. 1d and S2 show lattice fringes of 4.9 Å and 2.6 Å corresponding to the d spacing of (111) and (311) planes in Fe₃O₄, respectively, which further confirmed the XRD results. Good reducibility of iron oxides in iron-based FTO catalysts is well known to be essential to achieving high catalytic activity. Fe₃O₄ was more readily reducible compared to Fe₂O₃ reported in the art, and can be more efficiently transformed into Fe metal during the H₂ activation step and subsequently the active iron carbide phase upon syngas exposure under typical FTO reaction conditions.

Raman and X-ray photoemission spectroscopy (XPS) results illustrated that the CNS had multiple functional groups such as carboxyl, carbonyl and hydroxyl groups (FIG. S3). These functional groups have been proposed to contribute to catalyst performance by anchoring and stabilizing supported iron oxide nanoparticles. EDX mapping confirmed the presence of very evenly distributed K that was introduced during CNS synthesis by the potassium citrate precursor (FIG. S4). This ubiquitous and even distribution of K throughout the support plays a key role in the activity and performance of this catalyst system.

Prior to FTO reaction, FeₓO_{y}/CNS catalysts were reduced in H₂ for 3 hours at 400° C. to obtain metallic iron. In situ XRD confirmed the excellent reducibility of the FeₓO_{y}/CNS system, which formed FeO and α-Fe phases in 4% H₂ at 300° C., and further completely transformed to α-Fe metal at 400° C. (FIG. S5). Under FTO conditions, the metallic α-Fe was then readily carburized and formed the active species Fe₅C₂ (see below).

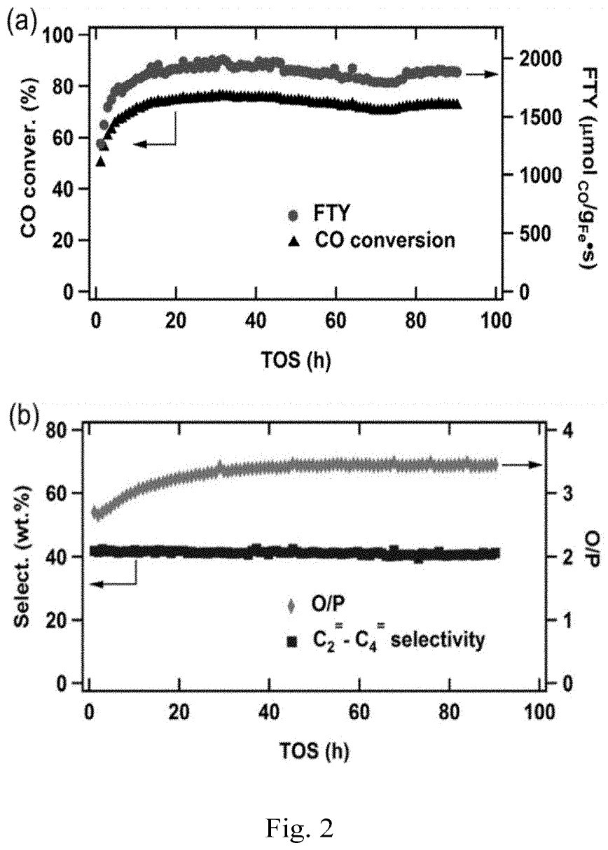

The CO conversion, iron time yield (FTY, the number of CO molecules converted to hydrocarbons per gram of Fe per second), light olefins selectivity and olefin to paraffin ratio (O/P) are illustrated in FIG. 2. In the first 10 h, the CO conversion quickly increased from 50% to 70%. This induction period corresponded to the carburization process that transformed metallic iron to catalytically active iron carbide phases. After 10 h of TOS, CO conversion slowly increased and subsequently reached a steady state value around ˜70%. The FeₓO_{y}/CNS catalysts demonstrated exceedingly high FTY values between 1790-1990 μmol_{CO}/g_{Fe}·s that were far superior to high performance known in the art, Fe-based carbon supported FTO or FT catalysts evaluated under similar reaction conditions. For example, FTY value of 29.8 μmol_{CO}/g_{F}e*s was reported for Fe₂O₃ on carbon nanofibers,³ 27.9 μmol_{CO}/g_{Fe}·s for Fe supported on N-doped carbon nanotubes, while commercial Ruhrchemie catalysts produced FTYs of 22.5 μmol_{CO}/g_{Fe}·s. The inventive FTYs are among the highest values achieved for iron based FTO and FT catalysts. Similarly impressive FTYs were recently reported in the art for Mg and K-promoted Fe on reduced graphene oxide catalyst, but in this study the activity decreased to 800-900 μmol_{CO}/g_{F}e·s after˜90 h of TOS. Additionally, FIG. 2b shows that the FeₓO_{y}/CNS catalyst exhibited good and stable selectivity towards C₂-C₄ olefins with a steady-state olefin to paraffin ratio (O/P) above 3. The reaction conditions were specifically chosen to favor short chain hydrocarbon production and essentially all of the products were C₁-C₅ molecules, with only trace amounts of hydrocarbons of C₆ and beyond.

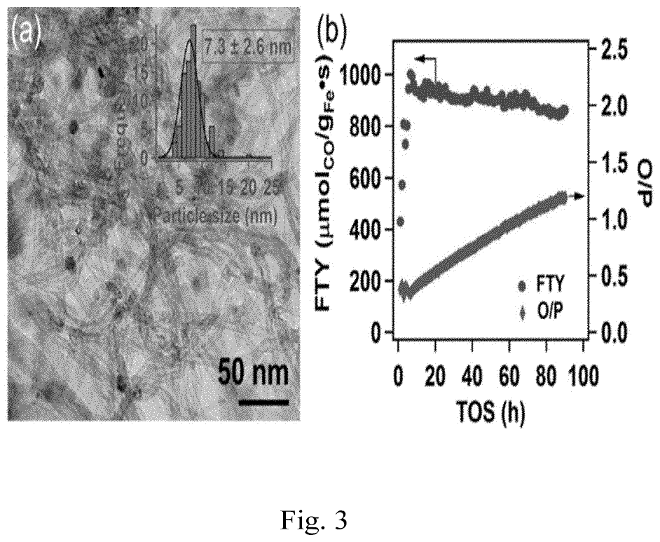

To evaluate the role of carbon support material, FeₓO_{y}/CNT catalyst was prepared and tested for FTO as a control sample. The as-received CNTs had an outer diameter of ˜10 nm and length of 3-20 μm. The average size of the CNT supported FeₓO_{y} nanoparticles was ˜ 7.3 nm (FIG. 3a) and XRD indicates the oxide was in the Fe₃O₄ phase (FIG. S6), which was the same starting phase as the FeₓO_{y}/CNS samples. Despite similar characteristics between the CNT and CNS supported samples, the FeₓO_{y}/CNS catalyst outperformed the FeₓO_{y}/CNT catalyst in all aspects of FTO synthesis (FIG. 3b, S8, Table 1). Although the CO conversion was relatively stable (˜45%) for FeₓO_{y}/CNT, it was significantly lower than that of FeₓO_{y}/CNS (˜70%), and the FTY slowly decreased from 1000 to 860 μmol_{CO}/g_{Fe}·s over 90 h TOS. Compared with the stable O/P ratio of 3.4 for the FeₓO_{y}/CNS catalyst, the FeₓO_{y}/CNT exhibited a substantially lower O/P ratio which also changed over time from 0.3 to 1.2 in 90 h (FIG. 3b and S8).

The catalytic performance of several Fe based catalysts is summarized in Table 1. To make a more direct comparison with the K-promoted CNS system, the activity of FeₓO_{y}/CNT promoted with 1% K (1K-FeₓO_{y}/CNT) was evaluated. While the 1K-FeₓO_{y}/CNT sample had better olefin selectivity (54.4%) than the unpromoted FeₓO_{y}/CNT (33.1%), the activity and stability of the 1K-FeₓO_{y}/CNT catalyst were drastically reduced, with a CO conversion of only 4.1% at 10 h TOS and almost complete deactivation after ˜18 h TOS. This finding validated that high FTO performance cannot be simply achieved by adding K onto CNTs and highlights the advantage of using CNS as a support. A standard non-supported sample, bulk Fe—Cu—K—SiO₂ catalyst promoted by Cu and K, achieved 52.3% CO conversion, but demonstrated poor stability after ˜18 h. Finally, compared with one of the best FTO catalysts known in the art, Fe₂O₃ supported on CNF, the 1882 μmol_{CO}/g_{F}e·s FTY value for the inventive FeₓO_{y}/CNS catalysts was ˜60 times higher and this activity was maintained with high CO conversion (72.6%) and high olefin selectivity (41.2%). The data and comparisons indicate that the K-promoted FeₓO_{y}/CNS system is one of the highest known performing Fe based FTO catalysts.

TABLE 1
Catalytic performance of FeₓO_{y}/CNS compared with FeₓO_{y}/CNT, 1 wt. % K promoted
FeₓO_{y}/CNT and other reference catalysts as measured by CO conversion, FTY and product
selectivity under the same FTO reaction conditions (350° C., 20 bar, H₂/CO = 1) except
Fe₂O₃/carbon nanofiber (CNF) (340° C., 20 bar, H₂/CO = 1).

| | | | | Selectivity (% wt.) | | | | |
|---|---|---|---|---|---|---|---|---|
| Sample | TOS (h) | CO (%) | FTY | CH₄ | C₂-C₄ | C₂^{═}-C₄^{═} | C₅⁺ | O/P |
| FeₓO_{y}/CNS | 90 | 72.6 | 1882 | 29.9 | 53.5 | 41.2 | 16.6 | 3.35 |
| FeₓO_{y}/CNT | 90 | 42.1 | 861 | 29.7 | 61.0 | 33.1 | 9.0 | 1.19 |
| 1K—FeₓO_{y}/CNT | 10 | 4.1 | 89.2 | 26.3 | 64.5 | 54.4 | 9.1 | 5.36 |
| Fe—Cu—K—SiO₂ | 18 | 52.3 | 161 | 47.1 | 46.5 | 26.0 | 6.4 | 1.27 |
| Fe₂O₃/CNF³ | 64 | 88 | 29.8 | 13 (% C) | 64 (% C) | 52 (% C) | 18 (% C) | 6.5 |

³FTY unit is μmol_{CO}/g_{Fe}•s. Note that the selectivity to C₂-C₄ includes both paraffins and olefins, whereas selectivity to C₂^{═}-C₄^{═} is specifically for olefins.

### Structure-Activity Correlation

Detailed characterization of the FTO catalysts has been carried out to understand the structure-activity correlation. The FeₓO_{y}/CNS catalysts undergo significant structural and phase changes under FTO reaction conditions (FIG. 4a, b). The post-reaction iron-based nanoparticles slightly increased to an average particle size of 12.1 nm (FIG. 4a inset), compared to 10.0 nm in fresh catalysts (FIG. 1b inset). Some larger nanoparticles (>30 nm) were also observed (FIG. 4a), indicative of some agglomeration.

Despite the occurrence of some sintering, both the catalytic activity and product selectivity were stable throughout the entire reaction testing process (FIG. 2). Analysis of the HRTEM images indicated post-reaction catalyst nanoparticles were composed of a Fe₅C₂ core with a thin amorphous iron oxide shell. The observed lattice fringes in the core were consistent with the d spacing of (510), (200), and (11-2) planes of Fe₅C₂ phase. In addition, the 700 angle also agreed with the expected angle between (021) and (−11-1) planes in Fe₅C₂ structure (FIG. 4b).

EDX mapping clearly illustrates the Fe₅C₂/amorphous iron oxide core/shell structure in the spent catalyst (FIG. S9): Fe was present in both the core and shell with C in the core and O in the shell. The same conclusion is further supported by XPS depth-profiling studies (FIG. S10). As the oxide shell was gradually removed by sputtering, the embedded Fe₅C₂ core became increasingly exposed as evidenced by the growth of the peak associated with Fe₅C₂ in the XPS spectra. The amorphous iron oxide shell may have resulted from exposing the post-reaction catalyst to air, or it may have formed in situ due to H₂O generation during FTO reaction.

Mössbauer spectroscopy had been utilized to study the evolution of FeₓO_{y} nanoparticles during the FTO process. The Mössbauer results also quantified the types of Fe species present in the catalyst before and after reaction (Table S1). The Mössbauer spectrum of the fresh FeₓO_{y}/CNS catalyst showed a sextet splitting pattern typically associated with magnetic iron species (FIG. 4c). Analysis of the spectrum (Table S1) suggests that Fe₃O₄ was the predominate phase in the fresh, unactivated, FeₓO_{y}/CNS catalyst, consistent with the XRD and HRTEM analysis.

In comparison, the Mössbauer spectrum of the spent catalyst showed significant differences (FIG. 4d). Simulation of the spectrum of the spent FeₓO_{y}/CNS catalyst (Table S1) showed that it was mainly composed of x-Fe₅C₂ (72% of the Fe species), with contributions from a minor FeₓC phase and a mixed oxide phase. The high x-Fe₅C₂ content in the spent catalyst correlated well with its high activity and selectivity.

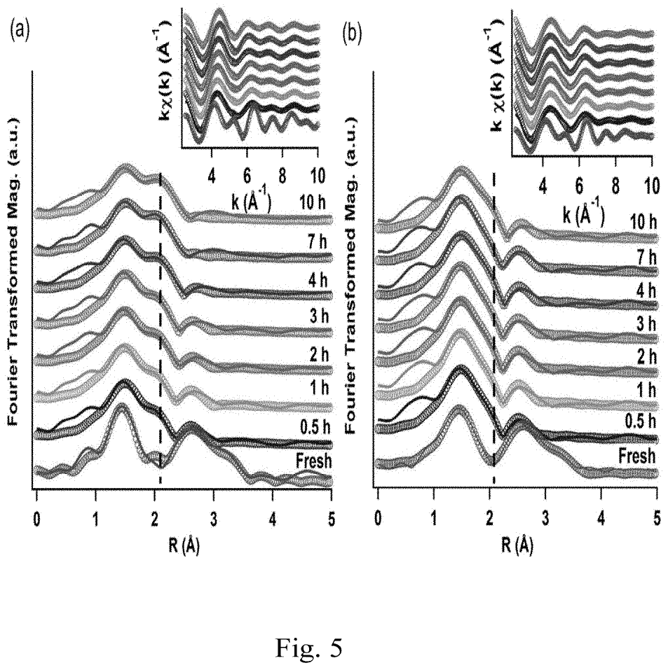

X-ray absorption spectroscopy (XAS) provides additional details on the transformation of iron oxide nanoparticles during FTO reactions. The evolution of the X-ray absorption near edge structure (XANES) spectra for both FeₓO_{y}/CNS and FeₓO_{y}/CNT catalysts at different reaction times demonstrated the conversion of iron oxide to iron carbide during FTO reaction (FIG. S11), consistent with TEM, XRD and Mössbauer results shown above. Despite the similar conversion processes for the two catalyst systems, the degree of carburization of FeₓO_{y}/CNS catalysts appeared to be much more complete than that of FeₓO_{y}/CNT catalysts. This difference is more clearly illustrated by Fourier transformed extended X-ray absorption fine structure (EXAFS) in FIG. 5. For the fresh catalysts, the first peak located between 1 and 2 Å is associated with the first Fe—O shell around the Fe absorbing core at 1.9-2.1 Å. The second peak between 2 and 3.5 Å originates from the Fe—Fe scattering of Fe₃O₄ at 3.0-3.5 Å (see fitting results in Table S2, S3). In the reacted catalysts, the Fe—Fe scattering from Fe₃O₄ in the fresh catalyst diminished as a function of TOS. Instead, a new peak at ˜ 2.0 Å appears (denoted by the vertical dashed lines in FIG. 5), which is ascribed to the Fe—Fe coordination from iron carbide composition (Table S4, S5). The growth of this Fe₅C₂ peak is much more pronounced for FeₓO_{y}/CNS catalysts (FIG. 5a) than FeₓO_{y}/CNT catalysts (FIG. 5b). These results suggest more effective carburization of iron oxide supported on CNS, leading to much improved catalytic performance for FeₓO_{y}/CNS catalysts compared with FeₓO_{y}/CNT catalysts (Table 1).

### Effect of Catalyst Support

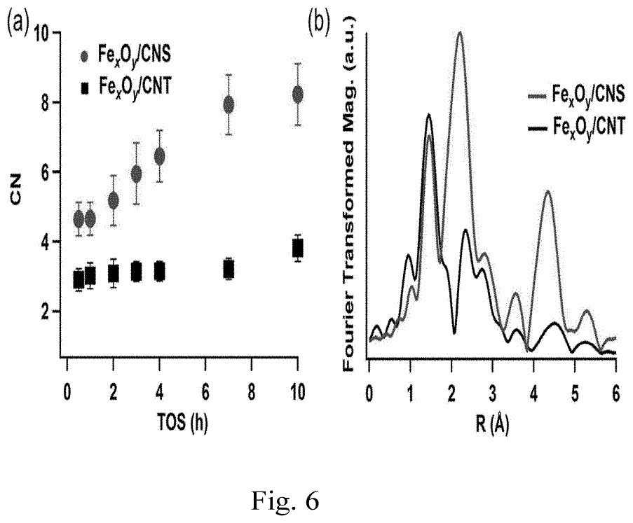

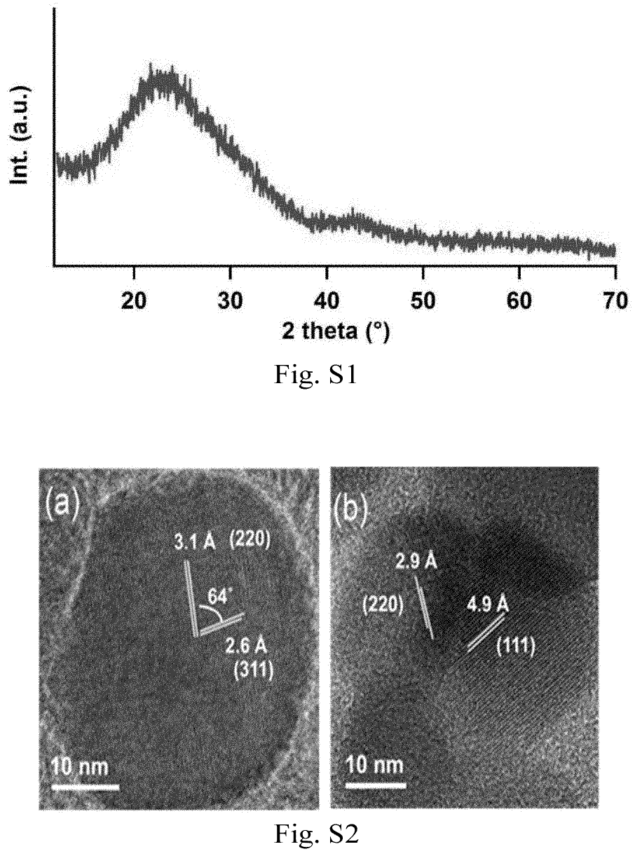

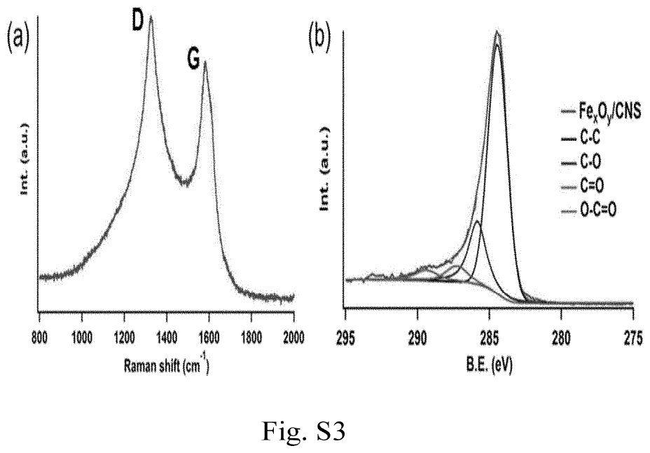

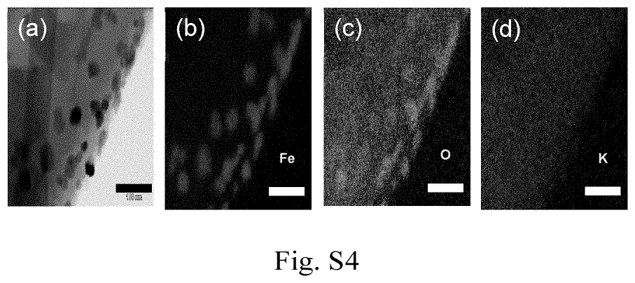

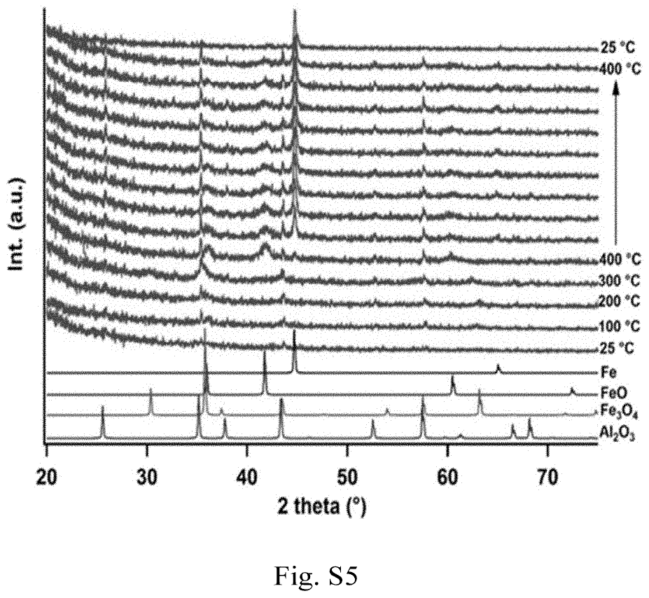

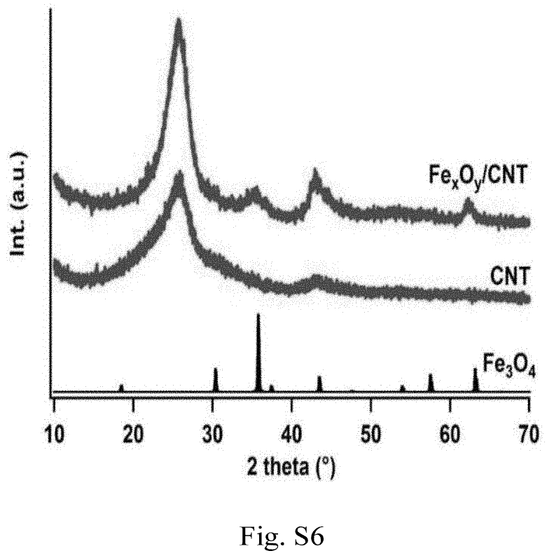

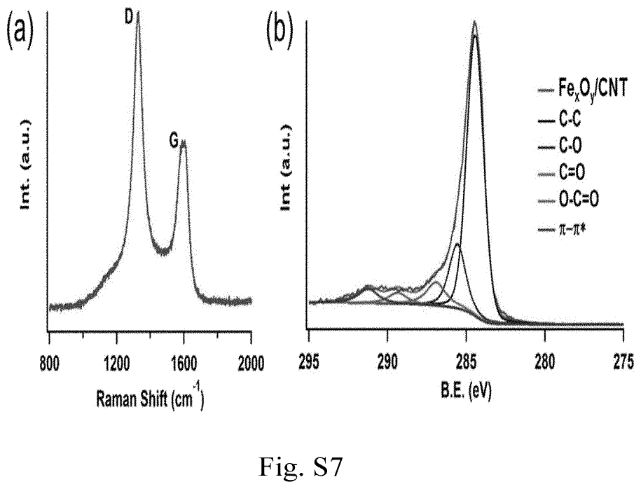

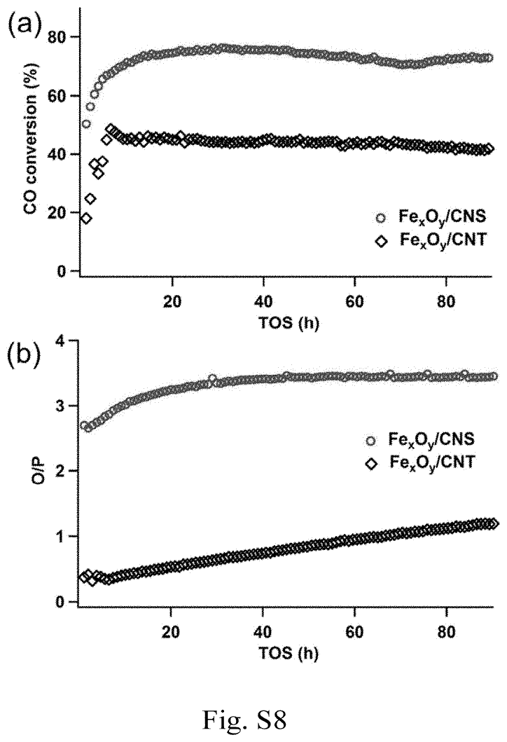

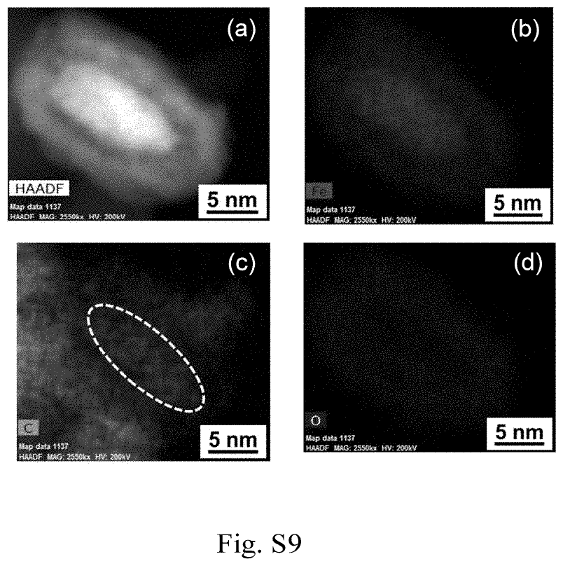

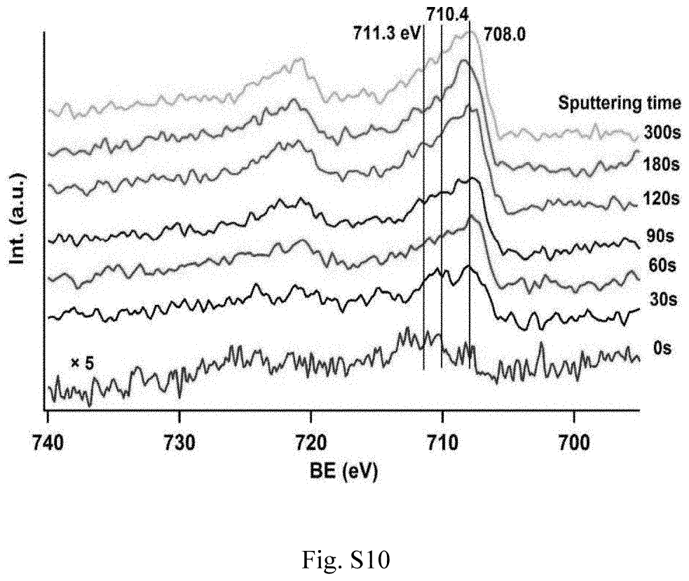

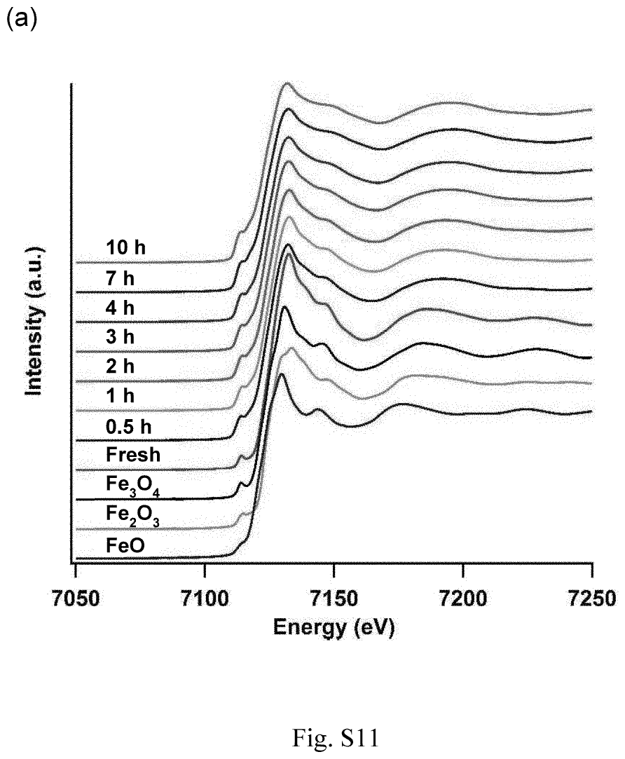

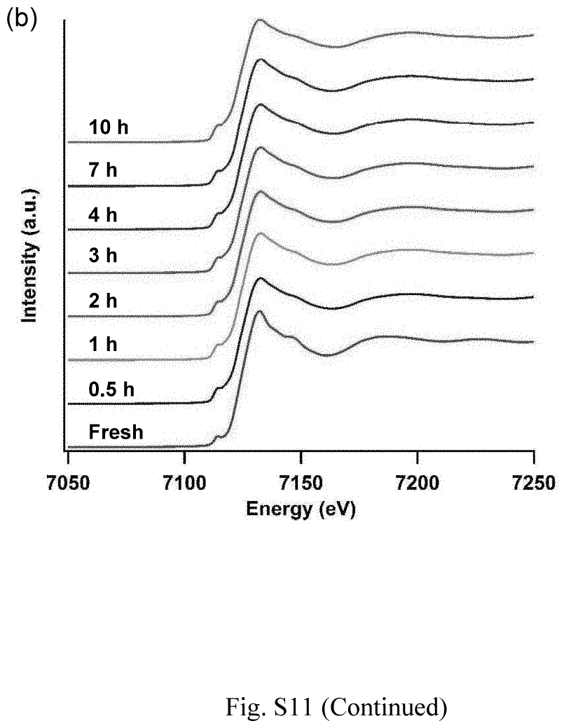

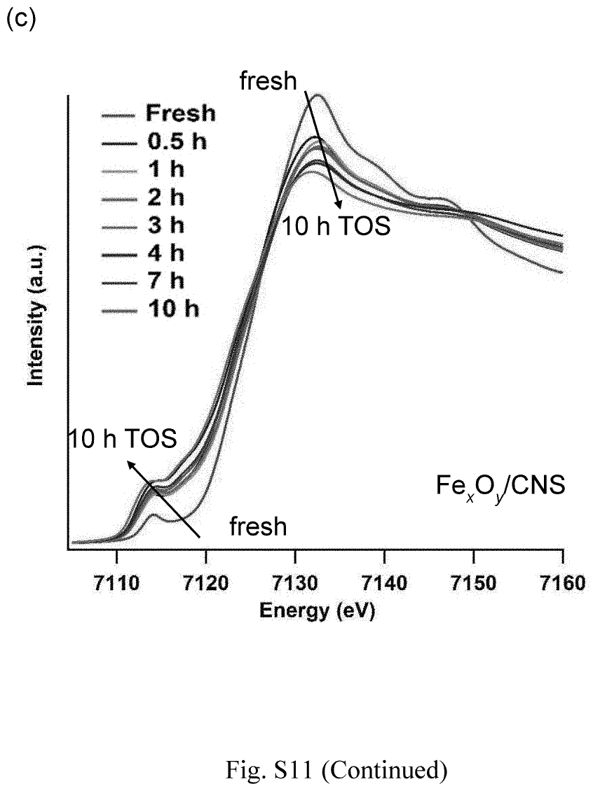

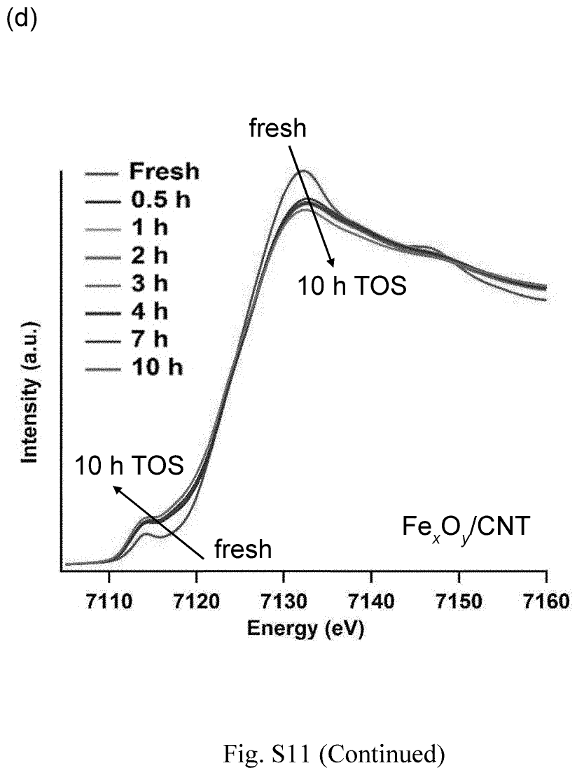

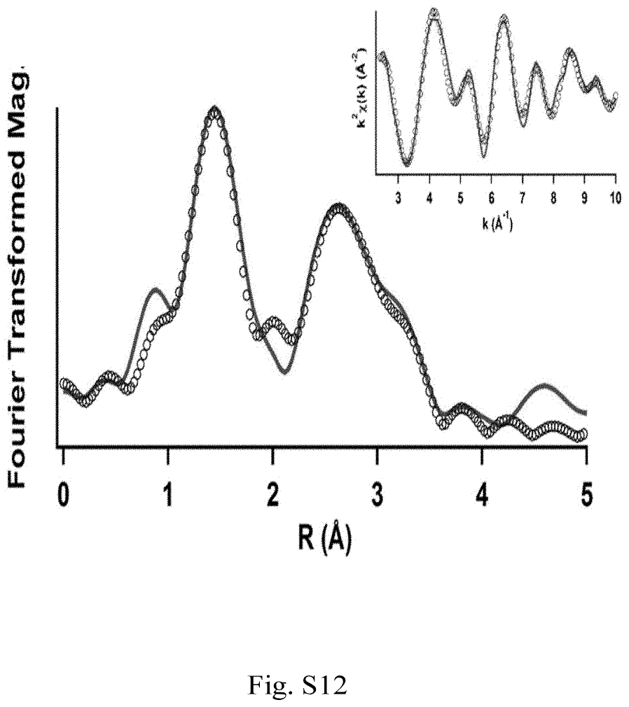

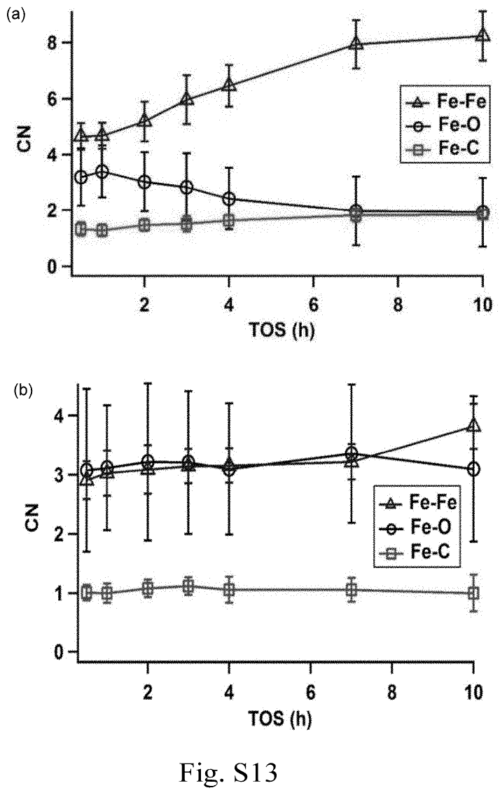

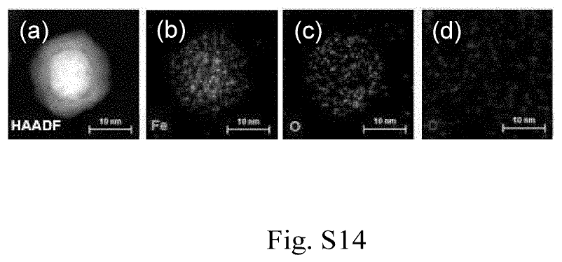

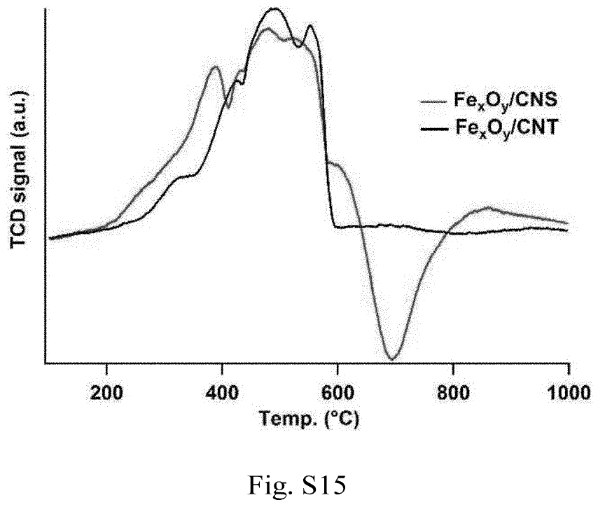

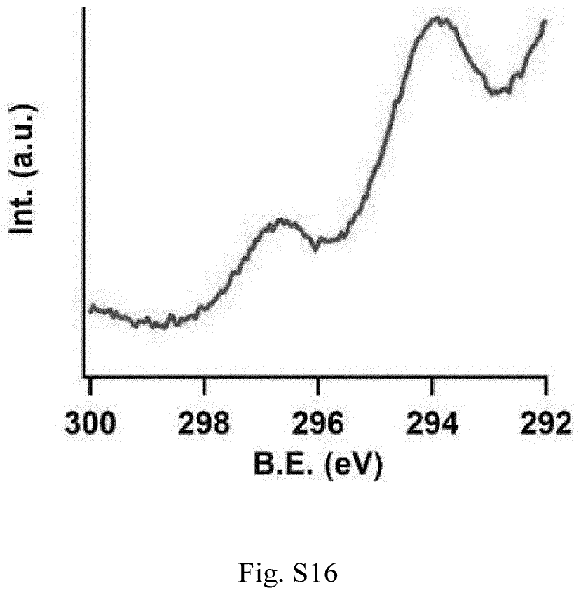

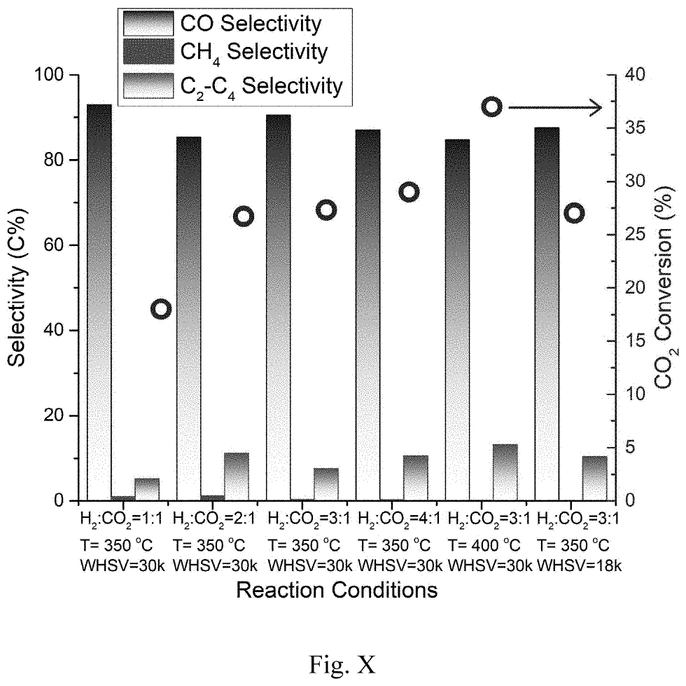

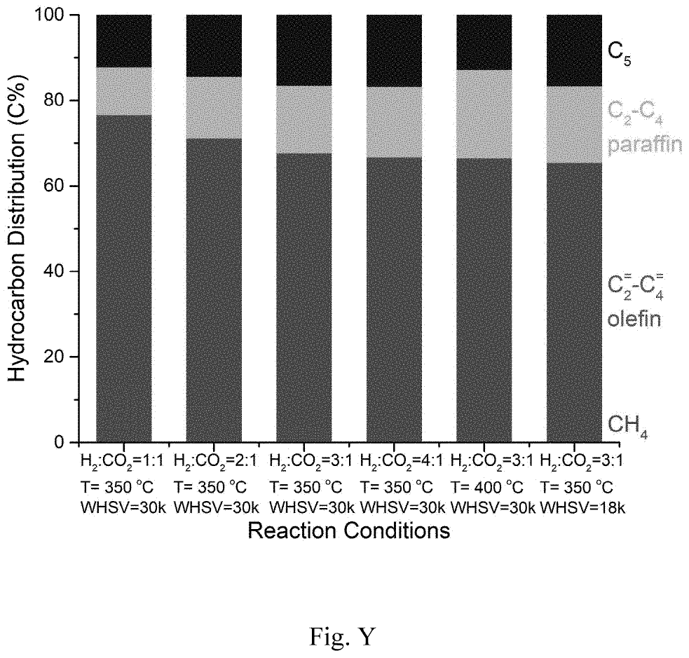

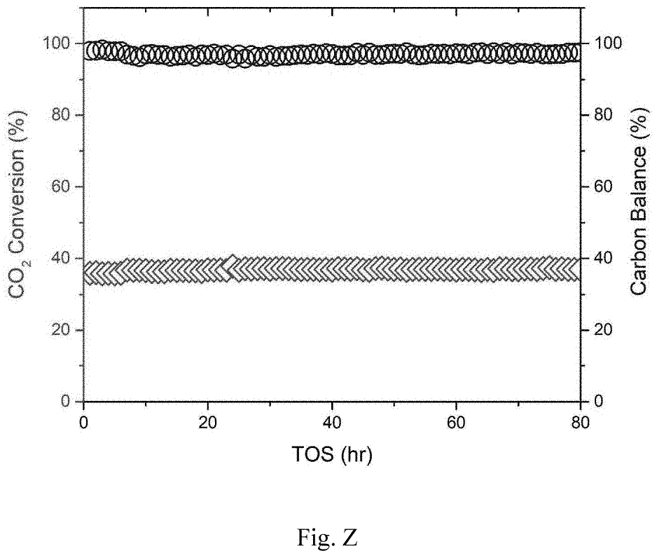

As discussed in the previous section, the difference in catalyst performance for CNS and CNT supported catalysts was a result of the more complete transformation from iron oxide to iron carbide for CNS supported catalysts. For quantitative comparison, the evolution of Fe—Fe EXAFS coordination number from Fe₅C₂ for FeₓO_{y}/CNS and FeₓO_{y}/CNT catalysts as a function of TOS is illustrated in FIG. 6a and FIG. S13. To understand the difference in carburization on different catalyst supports, the EXAFS spectrum of the H₂ reduced FeₓO_{y}/CNS catalyst (FIG. 6b) offers the direct evidence of the presence of metallic iron, whereas the H₂ reduced FeₓO_{y}/CNT catalyst contained mostly oxidized Fe species (FIG. 6b). Furthermore, EDX mapping of reduced FeₓO_{y}/CNS was consistent with a core/shell structure with a metallic Fe core and an amorphous iron oxide shell presumably due to exposure to air (FIG. S14). The improved reducibility of FeₓO_{y}/CNS catalyst is also suggested by the H₂-TPR profiles (FIG. S15).

These results suggest more robust formation of metallic Fe nanoparticles on the CNS support than on the CNT. The stabilization of metallic Fe nanoparticles by CNS subsequently leads to more effective carburization upon introduction of CO under FTO conditions. In contrast, for the reduced CNT supported catalyst, the less effective formation and/or stabilization of metallic iron entails that the conversion to iron carbides was hindered due to the extra barrier to push out oxygen in iron oxides. These different effects of the two support materials can explain the improved catalytic activity and selectivity for CNS supported catalysts.

A potential reason that CNS is a superior support material may be that CNS can offer an optimal interaction with the catalyst particles leading to enhanced reduction/carburization. The average particle size formed on CNSs is 10±5 nm but decreased to 7±3 nm on CNTs despite using identical synthesis procedures. This suggests a stronger interaction between the iron oxide nanoparticles and the CNT support resulting in the stabilization of smaller sized particles. This stronger interaction with the CNT support was consistent with the observation that the FeₓO_{y}/CNT catalyst was more difficult to reduce and carburize.

Another factor contributing to the catalytic performance of FeₓO_{y}/CNS was the potassium contained in the CNS support. Potassium is widely used as a promoter for improving olefin selectivity and activity by facilitating the formation of Hägg carbide, improving the surface CO/H₂ ratio, and stabilizing active iron facets. The CNS support was specifically chosen because it derives from carbonization of potassium citrate with residual K distributed evenly through the entire support (FIG. S4). The FeₓO_{y}/CNS catalyst contained 1.8 wt. % K in a partially oxidized state (FIG. S16) and offers a promoter effect. The K effect in FeₓO_{y}/CNT catalysts was evaluated by adding ˜ 0.1-1 wt. % K (1K-FeₓO_{y}/CNT, Table 1); however, these 1K-FeₓO_{y}/CNT catalysts had a much lower activity and reduced stability, despite an enhancement in light olefins selectivity. This result suggested the synergistic effect of the CNS support and the inherently embedded potassium evenly distributed on the CNS is more effective than simply adding K to CNT support.

K promoters are commonly used in various catalytic applications, such as ammonia synthesis. These K-promoted CNS supports should therefore have utility for a wide variety of catalyst applications beyond the current demonstration for FTO reactions.

### CO₂ Hydrogenation

CO₂ hydrogenation was examined with the K-promoted CNS supported FeₓO_{y} catalysts (FIG. X). Catalytic performance of the FeₓO_{y}/CNS catalyst for CO₂ hydrogenation was investigated using the following range of reaction conditions: T=350-400° C., P=20 bar, H₂/CO₂=1-4, WHSV=18,000-30,000 cm³ (STP)/(g_{cat}-h). CO₂ conversion increased with reaction temperature and H₂/CO₂ molar ratio of the feed gas. High CO₂ conversion up to 37% could be achieved at 400° C. and H₂/CO₂=3. CO was the main C-containing product under these reaction conditions, but small quantities of C₂-C₄ products were observed.

The C₁-C₅ hydrocarbon product distribution from CO₂ hydrogenation was characterized in more detail (FIG. Y). CO was excluded in this product distribution to better demonstrate that the selectivity to C₂-C₄ olefins can be tuned by optimizing key reaction parameters such as feed gas composition and space velocity. The FeₓO_{y}/CNS catalyst selectively converted CO₂ to C₂-C₄ olefins. Unwanted CH₄ production could be suppressed by increasing the H₂/CO₂ ratio of the feed gas or by lowering the reaction temperature.

The stability of the CO₂ hydrogenation activity of the K-promoted CNS supported FeₓO_{y} catalyst was also evaluated. FIG. Z shows catalytic performance as a function of time on stream (TOS) under the conditions of 400° C., 20 bar, H₂/CO₂=3 and WHSV=30,000 cm³ (STP)/(g_{ca}t-h). The catalyst exhibited stable performance for at least 80 h with no real evidence of degradation of performance. Additionally, the carbon balance remained close to 100% throughout the experiment illustrating the sample was not coking and/or that the reaction products were being accounted for, completely.

### CONCLUSIONS

New K-promoted CNS supported FeₓO_{y} catalysts for CO₂ hydrogenation and FTO synthesis have been developed. The catalyst demonstrated superior catalytic activity and stability, with good olefins selectivity. Its FTY in the range of 1790-1990 μmol_{CO}/g_{Fe}·s appeared to be the highest values of reported Fe based FTO or FT catalysts in the art. The catalyst was robust over ˜100 h of TOS. Moreover, the catalyst could be reused repeatedly without degradation in catalytic performance for at least 500 h cumulative TOS. The effect of the CNS support had been evaluated and compared with other carbon support materials such as CNT. EXAFS studies indicated that K-promoted CNS could stabilize the metallic iron nanoparticles during H₂ reduction, which enhanced the formation of iron carbide under FTO reaction conditions. The efficient and complete carburization of FeₓO_{y}/CNS catalyst resulted in its high catalytic activity, selectivity and stability. In contrast, the CNT supported catalyst nanoparticles exhibited smaller average sizes and were more difficult to reduce, leading to less efficient transformation to catalytically active iron carbide. These observations suggest that the K-promoted CNS support offers a relatively weak but balanced interaction with the catalyst nanoparticles that enables the improved catalyst reduction and carburization while maintaining the structural integrity under reaction conditions.

### Methods

### Catalyst Preparation

Carbon nanosheets (CNS) were prepared by carbonization of potassium citrate, which was heated in an alumina ceramic tube under N₂ to 850° C. with a ramp rate of 1° C./min and was held at this temperature for 1 h. This fabrication formulation and method were chosen to not only form interconnected CNS, but also to efficiently incorporate the K promoter into the catalyst support. The product was then cleaned with 10% HCl and subsequently washed with copious amounts of water until the solution pH was neutral. The carbon nanosheets were then dried at 70° C. for 2 h and further dried under vacuum for 12 h.

Ammonium iron citrate was used as the Fe precursor for depositing iron oxide nanoparticle catalysts on the CNS support. The nominal Fe content of all catalysts prepared in this study was fixed at 5 wt. %. Ammonium iron citrate solution (1.4 M, 333 μL) was diluted by 5 mL of water and added slowly to 500 mg of CNS until the powder was fully wet. The mixture was then allowed to dry slowly at 50° C. for several hours and further dried under vacuum overnight. Subsequently, the mixture was calcined at 500° C. for 2 h with a ramp rate of 5° C./min under N₂ to form the FeₓO_{y}/CNS catalyst.

For comparison, iron-based nanoparticles supported on carbon nanotubes (CNTs) were also prepared. Ammonium iron citrate solution was mixed with multiwalled carbon nanotubes (Sigma-Aldrich) with 6-13 nm in outer diameter and 2.5-20 μm in length and the resulting mixture was processed in the same manner as described above to form FeₓO_{y}/CNT catalyst. For K-promoted FeₓO_{y}/CNT catalyst, K₂CO₃ was added to ammonium iron citrate solution for the iron oxide deposition step. Elemental analysis of the blank CNT indicated that there is a trace amount (0.01 wt. %) of Fe in the as-received CNT.

The FeₓO_{y}/CNS catalyst were determined to contain 3.6 wt. % Fe and 1.8 wt. % K using ICP-MS. The unpromoted FeₓO_{y}/CNT contained 4.2 wt. % Fe and no K (below the ICP-MS detection limit). The nominal 1 wt. % K-promoted FeₓO_{y}/CNT contained 3.9 wt. % Fe and 1.2 wt. % K.

### Catalyst Performance Evaluation

CO₂ hydrogenation and FTO tests were conducted in a fixed-bed reactor system (Process Integral Development Engineered & Tech.). The prepared catalysts were evaluated at 350-400° C. and 20 bar, which favored the production of short chain hydrocarbons. The total gas flow rate was 100 cm³(STP)/min. For CO₂ hydrogenation, the weight hourly space velocity (WHSV) range was between 18,000 and 30,000 cm³(STP)/(g_{cat}·h). The CO₂ flow rate was fixed at 13 cm³(STP)/min while the H₂/CO₂ molar ratio was varied between 1 and 4. N₂ was used as a diluent and an internal standard. For FTO, the feed gas composition was CO/H₂/N₂=45/45/10 and the WHSV was 30,000 cm³(STP)/(g_{cat}·h). Prior to reaction, catalyst samples (200 mg) were activated in situ in flowing H₂ (50 cm³(STP)/min) at 400° C. and 1 bar for 3 h. The feed and product streams were analyzed online using an Agilent GC7890A equipped with flame ionization and thermal conductivity detectors (FID/TCD) as well as a methanizer. Separation of the compounds was performed using Ar as a carrier gas and 2 columns: molecular sieve 13× (6 ft×⅛ in. SS, 60/80 mesh) for light gases (H₂, N₂, CH₄, and CO) and Hayesep Q (10 ft×⅛ in. SS, 80/100 mesh) for CO₂ and C₂-C₆ hydrocarbons.

### Catalyst Characterization

Scanning transmission electron microscopy (STEM) images were taken with a Hitachi HD-2300A dedicated scanning transmission electron microscope with a field-emission gun (FEG) and an optimal resolution of 0.204 nm. The catalyst sample was crushed in an agate mortar and pestle and it was suspended in ethanol. Approximately 1-2 drops of the suspension were spread onto a Cu grid coated with a holey carbon film (HC—Cu grid). The grid was then dried in air. The bright-field imaging (BF), high-angle annular dark-filed (HAADF, or atomic number contrast, Z-contrast) imaging, and secondary electron imaging (SE) were carried out with a 200-kV electron probe. A Thermo Scientific Noran System SIX (NSS) energy dispersive X-ray spectroscopy (EDX) system was used to collect elemental chemistry and X-ray maps.

The atomic-resolution transmission electron microscopy (TEM) images of the catalysts were collected using an FEI Talos F200X instrument operated at 200 keV. With the aid of the ultra-bright field emitter, this instrument can image at near diffraction limit in annular dark-field STEM (ADF-STEM) mode and routinely achieve 1.4-1.5 angstrom resolution.

X-ray diffraction (XRD) measurements were carried out using a PANalytical X'pert pro X-ray diffractometer with Cu Kα radiation (X=1.5418 Å) with a step size of 0.017° and 200 s/step in the 2θ range from 10° to 80°. The XRD was operated at 45 kV and 40 mA. The crystallite size of Fe₃O₄ was calculated by the Scherer equation.

⁵⁷Fe Mössbauer spectra were collected using a ⁵⁷Co radiation source mounted on a velocity transducer operating under a constant acceleration mode. Velocity was calibrated with α-Fe metal. During the measurements, the samples were kept at 4.2 K in a SuperVaritemp dewar designed by Janis Research (Wilmington, Mass.). Mössbauer spectral simulations were performed by using the WMOSS software package (SEE Co., Edina, MN). Isomer shifts were quoted relative to α-Fe metal at 25° C.

X-ray absorption fine structure (XAFS) experiments were carried out at the 8-ID ISS beamline at Brookhaven National Laboratory's National Synchrotron Light Source II (NSLS-II) and the XAS beamline at Louisiana State University's Center for Advanced Microstructures and Devices (CAMD). Samples were prepared by mixing with boron nitride (BN) and were pressed into a pellet of ˜1 mm in thickness. Reference samples such as Fe₂O₃, Fe₃O₄ and FeO were mixed with BN with 5 wt. % Fe in BN. The pellets for FeₓO_{y}/CNS and FeₓO_{y}/CNT catalyst samples were prepared at a mass ratio of approximately 1:2 of catalyst:BN due to their low Fe concentration. For X-ray spectroscopy experiments, fresh catalysts, catalysts reduced under H₂ at 400° C. for 1 h, and catalysts undergoing FTO catalytic reaction conditions for different periods of time were prepared using a fixed-bed reactor under the same FTO conditions as the catalyst performance studies. All X-ray absorption measurements were conducted ex situ under ambient conditions. Fe K-edge XAFS data were collected in transmission mode for reference samples and in fluorescence mode for FeₓO_{y}/CNS and FeₓO_{y}/CNT samples. The IFEFFIT software package was used to analyze the XANES and EXAFS data to obtain the local structural information of iron. FEFF6 was applied to calculate single scattering paths modeled the χ(R).

Temperature-programmed reduction (TPR) was conducted in a Micromeritics Autochem 2950 HP chemisorption analyzer. Initially, samples (approximately 50 mg) were heated in flowing He at 300° C. for 30 min to remove moisture followed by cooling to room temperature. Subsequently, H₂-TPR was performed by heating the samples in flowing 10% H₂/Ar (50 cm³(STP)/min) from room temperature to 1100° C. with a ramp rate of 10° C./min. H₂ consumption during TPR was monitored by a thermal conductivity detector (TCD). A bath containing isopropyl alcohol (IPA) and liquid nitrogen was utilized during these measurements to trap water generated during H₂-TPR.

We claim:

1. A nanocatalyst, comprising:
a support structure, comprising:
a plurality of porous interconnected carbon nanosheets, and
a potassium promoter embedded in the carbon nanosheets; and
a plurality of iron oxide nanoparticles supported on the support structure.

2. The nanocatalyst of claim 1, wherein the iron oxide nanoparticles comprise Fe₃O₄.

3. A method of forming a nanocatalyst, comprising:
preparing a support structure, comprising:
interconnecting a plurality of porous carbon nanosheets; and
embedding a potassium promoter in the carbon nanosheets;
depositing a plurality of iron oxide nanoparticles on the support structure;
reducing the plurality of iron oxide nanoparticles to metallic iron; and
transforming the metallic iron into iron carbide.

4. The method of claim 3, wherein the depositing step comprises an iron precursor.

5. The method of claim 4, wherein the iron precursor comprises ammonium iron citrate.

6. The method of claim 3, wherein the preparing step comprises carbonization of potassium citrate.

7. The method of claim 3, wherein the reducing and transforming steps comprise reducing Fe₃O₄ nanoparticles to metallic iron nanoparticles and transforming to active Fe₅C₂, respectively.

8. The method of claim 7, wherein the reducing step comprises H₂ activation.

9. The method of claim 7, wherein the transforming step comprises exposing the metallic iron nanoparticles to syngas.

10. The nanocatalyst of claim 1, wherein said nanocatalyst is used in CO₂ hydrogenation and Fischer-Tropsch to Olefins synthesis.

11. The nanocatalyst of claim 10, wherein said nanocatalyst is reusable repeatedly without degradation in catalytic performance for at least 500 hours of cumulative TOS.

12. A method of preparing C₂-C₄ olefins, comprising:
fabricating a nanocatalyst, comprising:
preparing a support structure, comprising:
obtaining a plurality of carbon nanosheets;
interconnecting the plurality of carbon nanosheets;
carbonizing a potassium precursor; and
dispersing potassium promoter throughout the plurality of carbon nanosheets; and
depositing a plurality of iron oxide nanoparticles on the support structure;
initiating H₂ activation for reducing the plurality of iron oxide nanoparticles to metallic iron nanoparticles; and
exposing the metallic iron nanoparticles to carburization for transforming the metallic iron into active iron carbide.
